(19) United States
(12) Patent Application Publication
(10) Pub. No.: US 2026/0069205 A1
(43) Pub. Date: Mar. 12, 2026

(54) SYSTEMS AND METHODS FOR PHYSIOLOGICAL EVENT TRACKING

(71) Applicant: Apple Inc., Cupertino, CA (US)

(72) Inventors: Ainara GARDE MARTINEZ, Mountain View, CA (US); Lifeng MIAO, Menlo Park, CA (US); Siyi DENG, San Jose, CA (US); Vignesh KALIDAS, Sunnyvale, CA (US); Raghed EL BARDAN, San Jose, CA (US); Mohsen MOLLAZADEH, San Diego, CA (US); Matt T. BIANCHI, Santa Cruz, CA (US)

(21) Appl. No.: 19/320,924

(22) Filed: Sep. 5, 2025

Related U.S. Application Data
(60) Provisional application No. 63/692,148, filed on Sep. 8, 2024.

(51) Int. Cl. A61B 5/00; A61B 5/08; A61B 5/11; A61B 5/113

(52) U.S. Cl. CPC A61B 5/4818; A61B 5/08; A61B 5/1118; A61B 5/113; A61B 5/4812; A61B 5/7221; A61B 5/7264; A61B 5/7282; A61B 2560/0462; A61B 2562/0219

(57) ABSTRACT

A wearable device can include one or more sensors including one or more motion sensors. The data from the one or more motion sensors can include an indication of respiration, and the data can be processed to enable screening for physiological events, such as breathing disturbances associated with apnea/hypopnea events. In some examples, one or more features are extracted from the data. In some examples, to improve performance, one or more masks can be applied to the data from the one or more sensors. For example, the one or more masks can be used to perform processing on data from the one or more sensors when a user is determined to be sleeping and when the data from the one or more sensors is determined to be indicative of a quality respiration signal.

### CROSS-REFERENCE TO RELATED APPLICATIONS

This application claims the benefit of U.S. Provisional Application No. 63/692,148, filed Sep. 8, 2024, the content of which is herein incorporated by reference in its entirety for all purposes.

### FIELD

This relates generally to systems and methods for detecting, tracking and notifying a user of apneic events, and more particularly, to detecting, tracking and notifying a user of apneic events using a wearable device.

### BACKGROUND

Sound sleep is considered vital for health. Abnormal sleep habits may lead to many health disorders. Some sleep disorders, including sleep apnea, may adversely affect the physical and psychological functioning of the human body. Accordingly, providing users with information about potential apneic events (e.g., breathing disturbances) that occur while sleeping can be useful to improve sleep habits and health.

### SUMMARY

This relates to systems and methods for tracking sleep apnea related events using a wearable device. The wearable device can include one or more sensors including one or more motion (and/or orientation) tracking sensors (e.g., accelerometer, gyroscope, inertia-measurement unit (IMU), etc.), among other possible sensors. The one or more motion (and/or orientation) tracking sensors can include an indication of respiration and the data can be processed to enable screening for breathing disturbances (e.g., apnea/hypopnea events). The less-invasive screening (e.g., using the wearable device described herein) can provide determination and/or count of breathing disturbances that can be used as preliminary step before further, more-invasive steps (e.g., a sleep study with multiple sensor types including one or more electroencephalogram (EEG) sensors, airflow sensors, effort belt sensors, and/or pulse oximeters) are taken to determine breathing disturbances and/or diagnose a sleep apnea condition.

The data from the one or more sensors can be processed in the wearable device and/or by another device in communication with the one or more sensors of the wearable device to determine the likelihood that an apneic event occurred during a session and/or to count the number of likely apneic events during the session. In some examples, one or more features are extracted from the data, including one or more respiration features. In some examples, to improve performance, one or more masks can be applied to the data from the one or more sensors. For example, the one or more masks can be used to perform processing on data from the one or more sensors when a user is determined to be sleeping and when the data from the one or more sensors is determined to be indicative of a quality respiration signal.

### BRIEF DESCRIPTION OF THE DRAWINGS

FIGS. 1A-1B illustrate an example system that can be used to track breathing disturbances according to examples of the disclosure.

FIGS. 2A-2C illustrate example block and corresponding timing diagrams for breathing disturbance tracking according to examples of the disclosure.

FIG. 3 illustrates an example process for a rest/active classifier according to examples of the disclosure.

FIG. 4A illustrates an example block diagram of breathing disturbance tracking including feature extraction and mask generation for breathing disturbance classification according to examples of the disclosure.

FIG. 4B illustrates examples plots including plots illustrating motion data and aspects of breathing disturbance classification according to examples of the disclosure.

FIG. 5 illustrates an example process for a breathing disturbance classifier according to examples of the disclosure.

FIG. 6 illustrates an example process for a quality check classifier according to examples of the disclosure.

FIGS. 7A-7B illustrate a block diagram 700 for in-bed transition detection and a plot 720 indicative of in-bed transition detection according to examples of the disclosure.

FIG. 8 illustrates a timing diagram corresponding to the operations for generating the second mask according to examples of the disclosure.

### DETAILED DESCRIPTION

This relates to systems and methods for tracking sleep apnea related events using a wearable device. The wearable device can include one or more sensors including one or more motion (and/or orientation) tracking sensors (e.g., accelerometer, gyroscope, inertia-measurement unit (IMU), etc.), among other possible sensors. The one or more motion (and/or orientation) tracking sensors can include an indication of respiration and the data can be processed to enable screening for breathing disturbances (e.g., apnea/hypopnea events, often referred to as apneic events for brevity). The less-invasive screening (e.g., using the wearable device described herein) can provide determination and/or count of breathing disturbances that can be used as preliminary step before further, more-invasive steps (e.g., a sleep study with multiple sensor types including one or more electroencephalogram (EEG) sensors, airflow sensors, effort belt sensors, and/or pulse oximeters) are taken to determine breathing disturbances and/or diagnose a sleep apnea condition.

The data from the one or more sensors can be processed in the wearable device and/or by another device in communication with the one or more sensors of the wearable device to determine the likelihood that an apneic event occurred during a session and/or to count the number of occurrences of likely apneic events during the session. In some examples, one or more features are extracted from the data, including one or more respiration features. In some examples, to improve performance, the one or more features include one or more time-domain respiration features from one selected axis (or more than one) of a multi-axis stream of the motion data, and/or include one or more frequency domain features from the multi-axis stream of the motion data. In some examples, to improve performance, one or more masks can be applied to the data from the one or more sensors. For example, the one or more masks can be used to perform processing on data from the one or more sensors when a user is determined to be sleeping and when the data from the one or more sensors is determined to be indicative of a quality respiration signal (e.g. masking out or excluding other data when the user is determined to be awake or when the data from the one or more sensors is determined to be indicative of a non-quality respiration signal).

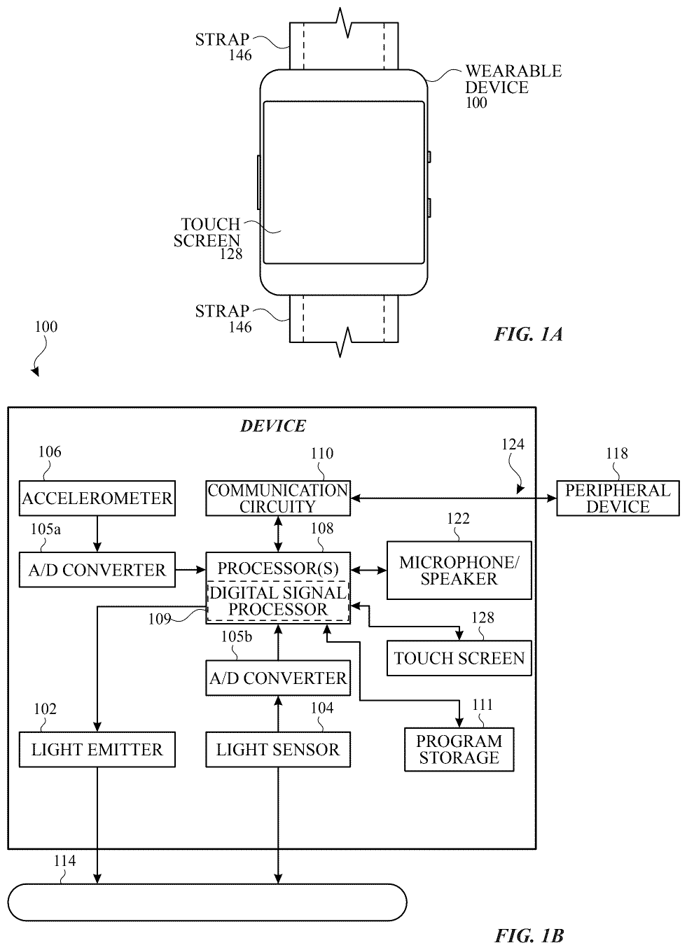

FIG. 1A illustrates an example wearable device 100 that can be attached to a user using a strap 146 or another fastener. Wearable device 100 can include one or more sensors, the data from which can be used to determine that a sleep disturbance has occurred and to count a number of occurrences of breathing disturbances within one or more sleep sessions. Optionally, wearable device 100 can include a touch screen 128 to display the results of breathing disturbance tracking as described herein. Additionally it is understood that although the wearable device 100 of FIG. 1A-1B includes a touch screen, determining a breathing disturbance or counting breathing disturbances in a session can be implemented using devices without a touch screen or a display.

In some examples, as shown in FIG. 1A, wearable device 100 is a wrist-worn device (e.g., a watch, wristband, etc.). It is understood that the wearable device is not limited as a wrist-worn device, and may be wearable in another manner (e.g., in proximity or affixed to other body parts such as one or more digits, head, chest, back, ankle, etc.). It is understood that although primarily described as a wearable device for improved user experience and performance, that the device including sensors can be a non-wearable device.

FIG. 1B illustrates an example block diagram of the architecture of wearable device 100 used to track breathing disturbances according to examples of the disclosure. As illustrated in FIG. 1B, the wearable device 100 can include a one or more sensors. For example, the wearable device 100 can optionally include an optical sensor including one or more light emitters 102 (e.g., one or more light emitting diodes (LEDs)) and one or more light sensors 104 (e.g., one or more photodetectors/photodiodes). The one or more light emitters can produce light in ranges corresponding to infrared (IR), green, amber, blue and/or red light, among other possibilities. The optical sensor can be used to emit light into a user's skin 114 and detect reflections of the light back from the skin. The optical sensor measurements by the one or more light sensors can be converted to digital signals (e.g., a time domain photoplethysmography (PPG) signal) for processing via an analog-to-digital converter (ADC) 105b. The optical sensor and processing of optical signals by the one or more processors 108 can be used, in some examples, for various functions including estimating physiological characteristics (e.g., heart rate, arterial oxygen saturation, etc.) or detecting contact with the user (e.g., on-wrist/off-wrist detection).

The one or more sensors can include a motion-tracking and/or orientation-tracking sensor such as an accelerometer, a gyroscope, an inertia-measurement unit (IMU), etc. For example, the wearable device 100 can include accelerometer 106 that can be a multi-channel accelerometer (e.g., a 3-axis accelerometer). As described in more detail herein, the motion-tracking and/or orientation-tracking sensor can be used to extract motion and respiration features used to determine the likelihood that a breathing disturbance has occurred and/or to count the number of breathing disturbances in one or more sessions. Measurements by accelerometer 106 can be converted to digital signals for processing via an ADC 105a.

The wearable device 100 can also optionally include other sensors including, but not limited to, a photothermal sensor, a magnetometer, a barometer, a compass, a proximity sensor, a camera, an ambient light sensor, a thermometer, a global position system sensor, and various system sensors which can sense remaining battery life, power consumption, processor speed, CPU load, and the like. Although various sensors are described, it is understood that fewer, more, or different sensors may be used.

The data acquired from the one or more sensors (e.g., motion data, optical data, etc.) can be stored in memory in wearable device 100. For example, wearable device 100 can include a data buffer (or other volatile or non-volatile memory or storage) to store temporarily (or permanently) the data from the sensors for processing by processing circuitry. In some examples, volatile or non-volatile memory or storage can be used to store partially processed data (e.g., filtered data, down-sampled data, extracted features, etc.) for subsequent processing or fully processed data for storage of sleep tracking results and/or display or reporting sleep tracking results to the user.

The wearable device 100 can also include processing circuitry. The processing circuitry can include one or more processors 108. One or more of the processors can include a digital signal processor (DSP) 109, a microprocessor, a central processing unit (CPU), a programmable logic device (PLD), a field programmable gate array (FPGA), and/or the like. In some examples, the wearable device 100 can include a host processor and a low-power processor. The low-power processor may be a continuously powered processor and the host processor may be powered up or powered down depending on a mode of operation. For example, a low-power processor can sample accelerometer 106 while a user is sleeping (e.g., when the host processor may be powered off), whereas the host processor can optionally perform some or all of the processing including determining a likelihood of a breathing disturbance and/or counting a number of breathing disturbance at the conclusion of the sleep session (e.g., when the host processor may be powered on). The various processing and classifiers described in more detail herein can be implemented entirely in the low-power processor, entirely in the host processor, or implemented partially in both the low-power processor and the host processor.

In some examples, some of the sensing and/or some of the processing can be performed by a peripheral device 118 in communication with the wearable device. The peripheral device 118 can be a smart phone, media player, tablet computer, desktop computer, laptop computer, data server, cloud storage service, or any other portable or non-portable electronic computing device (including a second wearable device). The peripheral device may include one or more sensors (e.g., a motion sensor, etc.) to provide input for one of the classifiers described herein and processing circuitry (e.g., the same or similar to one or more processors 108) to perform some of the processing functions described herein (e.g., using instructions stored in memory of wearable device 100 and/or peripheral device 118). Wearable device 100 and peripheral device 118 can also include communication circuitry 110 to communicatively couple to the peripheral device 118 via wired or wireless communication links 124. For example, the communication circuitry 110 can include circuitry for one or more wireless communication protocols including cellular, Bluetooth, Wi-Fi, etc.

In some examples, wearable device 100 can include a touch screen 128 to display the breathing disturbance tracking results (e.g., displaying information corresponding to potential apneic events, and/or number of apneic events) and/or to receive input from a user. In some examples, touch screen 128 may be replaced by a non-touch sensitive display or the touch and/or display functionality can be implemented in another device. In some examples, wearable device 100 (and/or peripheral device 118) can include a microphone/speaker 122 for audio input/output functionality, haptic circuitry to provide haptic feedback to the user, and/or other sensors and input/output devices. Wearable device 100 (and/or peripheral device 118) can also include an energy storage device (e.g., a battery) to provide a power supply for the components of wearable device 100.

The one or more processors 108 (also referred to herein as processing circuitry) can be connected to program storage 111 and can be configured to (programmed to) to execute instructions stored in program storage 111 (e.g., a non-transitory computer-readable storage medium). The processing circuitry, for example, can provide control and data signals to generate a display image on touch screen 128, such as a display image of a user interface (UI), optionally including results for a breathing disturbance tracking session. The processing circuitry can also receive touch input from touch screen 128. The touch input can be used by computer programs stored in program storage 111 to perform actions that can include, but are not limited to, moving an object such as a cursor or pointer, scrolling or panning, adjusting control settings, opening a file or document, viewing a menu, making a selection, executing instructions, operating a peripheral device connected to the host device, answering a telephone call, placing a telephone call, terminating a telephone call, changing the volume or audio settings, storing information related to telephone communications such as addresses, frequently dialed numbers, received calls, missed calls, logging onto a computer or a computer network, permitting authorized individuals access to restricted areas of the computer or computer network, loading a user profile associated with a user's preferred arrangement of the computer desktop, permitting access to web content, launching a particular program, encrypting or decoding a message, and/or the like. The processing circuitry can also perform additional functions that may not be related to touch processing and display. In some examples, processing circuitry can perform some of the signal processing functions (e.g., classification) described herein.

Note that one or more of the functions described herein, including breathing disturbance tracking, can be performed by firmware stored in memory or instructions stored in program storage 111 and executed by the processing circuitry. The firmware can also be stored and/or transported within any non-transitory computer-readable storage medium for use by or in connection with an instruction execution system, apparatus, or device, such as a computer-based system, processor-containing system, or other system that can fetch the instructions from the instruction execution system, apparatus, or device and execute the instructions. In the context of this document, a “non-transitory computer-readable storage medium” can be any medium (excluding signals) that can contain or store the program for use by or in connection with the instruction execution system, apparatus, or device. The computer-readable storage medium can include, but is not limited to, an electronic, magnetic, optical, electromagnetic, infrared, or semiconductor system, apparatus or device, a portable computer diskette (magnetic), a random access memory (RAM) (magnetic), a read-only memory (ROM) (magnetic), an erasable programmable read-only memory (EPROM) (magnetic), or flash memory such as compact flash cards, secured digital cards, universal serial bus (USB) memory devices, memory sticks, and the like.

The firmware can also be propagated within any transport medium for use by or in connection with an instruction execution system, apparatus, or device, such as a computer-based system, processor-containing system, or other system that can fetch the instructions from the instruction execution system, apparatus, or device and execute the instructions. In the context of this document, a “transport medium” can be any medium that can communicate, propagate, or transport the program for use by or in connection with the instruction execution system, apparatus, or device. The transport medium can include, but is not limited to, an electronic, magnetic, optical, electromagnetic, or infrared wired or wireless propagation medium.

It should be apparent that the architecture shown in FIG. 1B is only one example architecture, and that the wearable device 100 (and/or peripheral device 118) could have more or fewer components than shown, or a different configuration of components. The various components shown in FIG. 1B can be implemented in hardware, software, firmware, or any combination thereof, including one or more signal processing and/or application specific integrated circuits. Additionally, the components illustrated in FIG. 1B can be included within a single device or can be distributed between multiple devices.

Conventionally, diagnosing sleep apnea requires a patient to spend one or more nights in a sleep laboratory (or at home with a home sleep apnea testing kit), wearing various sensors including electroencephalogram sensors, airflow sensors, respiratory effort sensors and a pulse oximetry sensor. The use of wearable device 100 can provide an early screening for breathing disturbances associated undiagnosed sleep apnea without the more invasive sensors required for a proper diagnosis. If determinations of one or more breathing disturbances during one or more sessions satisfy one or more criteria (e.g., a threshold number of breathing disturbances per session, a threshold number of sessions, etc.) corresponding to an undiagnosed sleep apnea for a user, the user can then seek attention of a healthcare or medical provider and undergo more invasive testing to diagnose the sleep apnea condition.

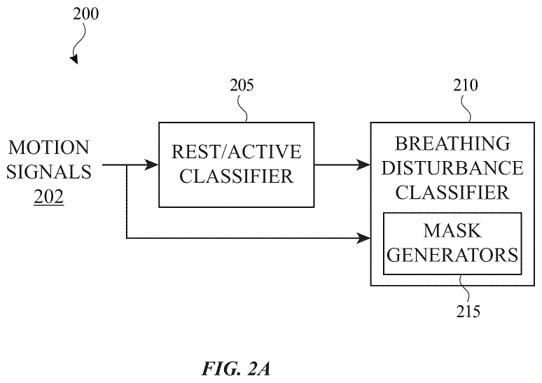

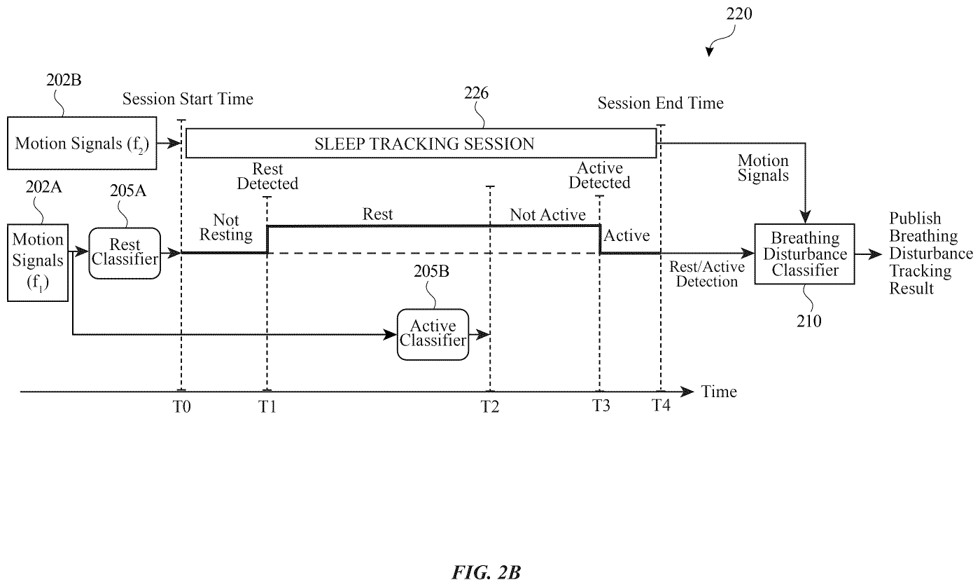

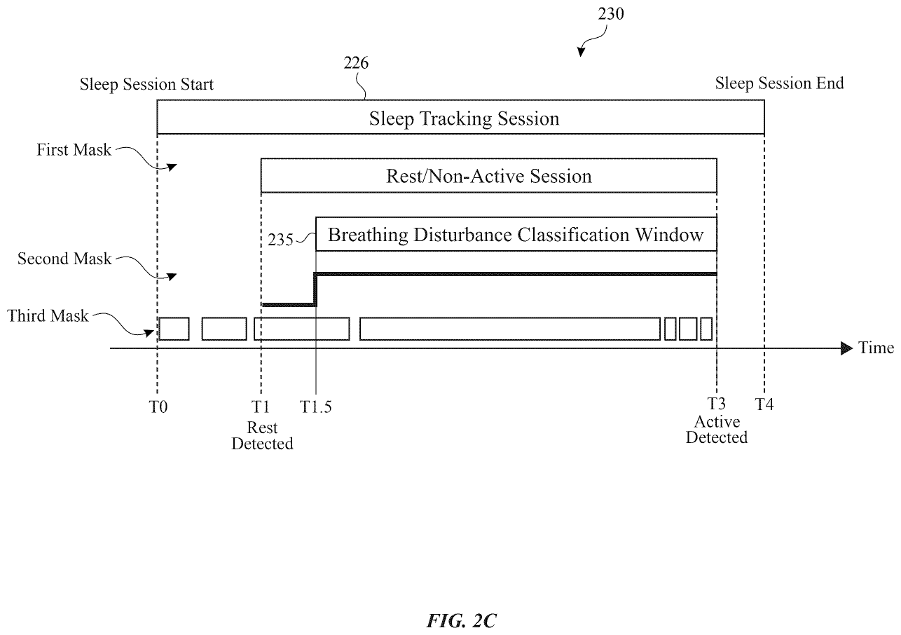

FIGS. 2A-2C illustrate example block and corresponding timing diagrams for breathing disturbance tracking according to examples of the disclosure. In some examples, breathing disturbance tracking can be implemented in conjunction with sleep tracking for one or more sleep states (e.g., awake, rapid eye movement (REM) sleep, or one or more non-REM sleep stages). FIG. 2A illustrates an example block diagram 200 of processing circuitry for breathing disturbance tracking according to examples of the disclosure. The processing circuitry can include a digital signal processor (e.g., corresponding to DSP 109 in FIG. 1B) and/or one or more additional processors (e.g., corresponding to one or more processors 108). In some examples, the processing circuitry can include a programmable logic device (PLD), field programmable gate array (FPGA), or other logic device. The processing circuitry can include or otherwise implement a rest/active classifier 205, a breathing disturbance classifier 210, and one or more mask generators 215. The classifications and/or mask generation can be based on motion signals 202, such as from one or more motion sensors (e.g., one or more accelerometers or a multi-axis accelerometer 106). The classifications and/or mask generation can be implemented in hardware, software, firmware, or any combination thereof.

Rest/active classifier 205 can be optionally included as part of breathing disturbance tracking to bound aspects of the data to be stored and/or processed for breathing disturbance classification (potentially reducing the storage and/or processing requirements and power consumption for the breathing disturbance tracking system). In particular, the rest/active classifier 205 can be used to define a start time corresponding to a classification that a user is resting and/or an end time corresponding to a classification that the user is active and not resting or sleeping. The bounding of aspects of the breathing disturbance classification to such a rest (non-active) session between the start time and the end time assumes that a user is unlikely to be sleeping (and therefore unlikely to have a breathing disturbance) while active/not-resting. In some examples, the rest/active classifier 205 can be implemented as one or more classifiers (e.g., a separate rest classifier 205A and a separate active classifier 205B). In some examples, the same classifier can be used but different thresholds can be used for rest classification before the start time than used for active classification after the start time.

FIGS. 2B-2C illustrates example timing diagram 220 and timing diagram 230 illustrating features and operation of the processing circuitry for breathing disturbance tracking according to examples of the disclosure. Timing diagram 220 illustrates features and operation of the rest/active classification with respect to acquiring motion signals 202, and the breathing disturbance classification based on the motion signals 202 and output of the rest/active classification. Timing diagram 230 illustrates features and operation of mask generation for breathing disturbance classification.

At time T0, the rest classifier 205A (e.g., the rest/active classifier using the “rest” thresholding parameters) can be triggered and can begin processing input data (e.g., motion signals 202A corresponding to motion signals 202 from motion sensors, optionally with some pre-processing, such as filtering, down-sampling, etc.) in accordance with process 300 to detect whether a user is resting or not (e.g., in a rest state or active state). In some examples, the rest classification can begin in response to satisfaction of one or more first triggering criteria. The one or more first triggering criteria can include a first trigger criterion that is met at a pre-defined time or in response to a user input. For example, the rest classifier can be triggered at a user-designated “bedtime” (or a default bedtime if the sleep tracking feature is enabled for the system without the user designating a bedtime) or a predefined time (e.g., 120 minutes, 90 minutes, 60 min, 45, minutes, 30 minutes, etc.) before the user-designated bedtime (or default bedtime). In some examples, the rest classifier can be triggered by a user request to perform a sleep tracking session and/or a breathing disturbance tracking session (or an indication that the user is currently in-bed or plans to go to bed soon). In some examples, in addition to the first trigger criterion, the rest classifier can process input only after an indication that the wearable device is worn by the user (or the absence of an indication that the wearable device is not off-wrist). For example, the one or more first triggering criteria can further include a second criterion that is satisfied when detecting that the wearable device is on-wrist (e.g., using the optical sensor or other sensor). The one or more first triggering criteria can further include a third criterion that is satisfied when detecting that the wearable device is not charging (e.g., via an inductive charger). Although three example criteria are described, it is understood that fewer, more, or different criteria can be used in some examples. In some examples, the rest classifier can process data until the rest classifier indicates that the user is in a rest state (at T1). In some examples, T1 can define the start time of a session (e.g., a rest/non-active session described below with respect to a first mask). In some examples, the rest classifier can process data until a timeout occurs, at which time sleep and/or breathing disturbance tracking can be terminated.

In some examples, at time T2, an active classifier 205B (e.g., the rest/active classifier using the “active” thresholding parameters) can be triggered and can begin processing input data (e.g., motion signals 202A corresponding to motion signals 202 from motion sensors, optionally with some pre-processing, such as filtering, down-sampling, etc.) in accordance with process 300 to detect whether the user is active or not (e.g., in an active state or a rest state). In some examples, the active classifier can begin in response to satisfaction of one or more second triggering criteria. The one or more second triggering criteria can include a first trigger criterion that is met at a pre-defined time or in response to a user input. For example, the active classifier can be triggered at a user-designated “wake-up time” (or a default wake-up time) or a predefined time (e.g., 120 minutes, 90 minutes, 60 min, 45, minutes, 30 minutes, etc.) before a user-designated “wake-up time” (or default wake-up time). In some examples, the active classifier can process data until the active classifier indicates that the user is in an active state. In some examples, after the active state is indicated by the active classifier, the user can be presented with a notification and the user input in response (e.g., tapping a button on the touch screen of the wearable device) can confirm the active state. In some examples, the active state (and its confirmation via user input, if implemented) can define the end time of the session (e.g., a rest/non-active session described below with respect to a first mask). In some examples, the end time of the session can be defined as a threshold period of time after the transition to the active state is indicated. As illustrated in FIG. 2B, T3 can define the end time of a session.

In some examples, the session can be terminated in other ways. In some examples, the session can be terminated upon dismissal of an alarm, detecting that the wearable device is off-wrist (e.g., using the optical sensor or other sensor), detecting that the wearable device is charging, a session timeout (e.g., a threshold time after T1 or after a threshold time after a user-designated wake-up time), a user input to end a session, or detecting an active state classification by the active classifier after a user-designated wake-up time, among other possibilities.

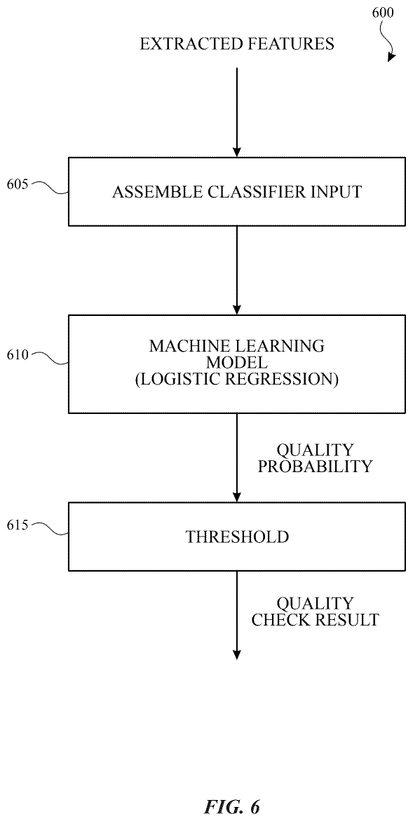

As shown in FIGS. 2B-2C, the sleep tracking session 226 can be defined between TO and T4, which can define the bounds of relevant motion signal data collection for use in mask generation and breathing disturbance classification. Ultimately, a subset of the data (e.g., motion signals 202) collected during the sleep tracking session 226 is used for breathing disturbance classification, which is shown as the breathing disturbance classification window 235. The subset of the data can be achieved using one or more masks. For example, a first mask can correspond to a rest (non-active) session, as shown in FIGS. 2B-2C, defined by the start time T1 and the end time T3. A second mask, shown in FIG. 2C, can correspond to the transition at time T1.5 from a non-sleep state to a sleep state (after the transition to the rest state), which can remove non-sleep rest portions of the rest session (between T1 and T1.5) from the breathing disturbance classification window 235. A third mask, shown in FIG. 2C, can correspond to a signal quality mask, to exclude portions of the data that are inaccurate due to motion artifacts and off-wrist conditions. As described herein, the third mask can eliminate segments of the data within breathing disturbance classification window 235 with poor signal quality. Additional details regarding masking are described with respect to FIGS. 6-8 As described herein, a session in some contexts refers a period between T1 and T3 (e.g., a rest period corresponding to a rest mask described herein) and in some contexts refers to a period beginning before T1 and/or ending after T3 (e.g., between TO and T4, between T1 and T4, between TO and T3) including data before and/or after the rest period corresponding to a sleep tracking session.

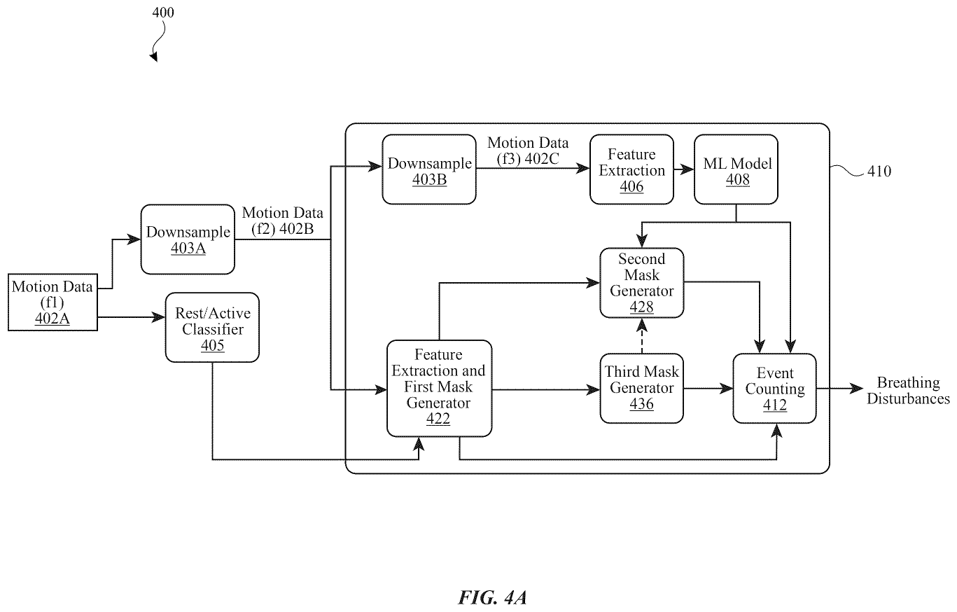

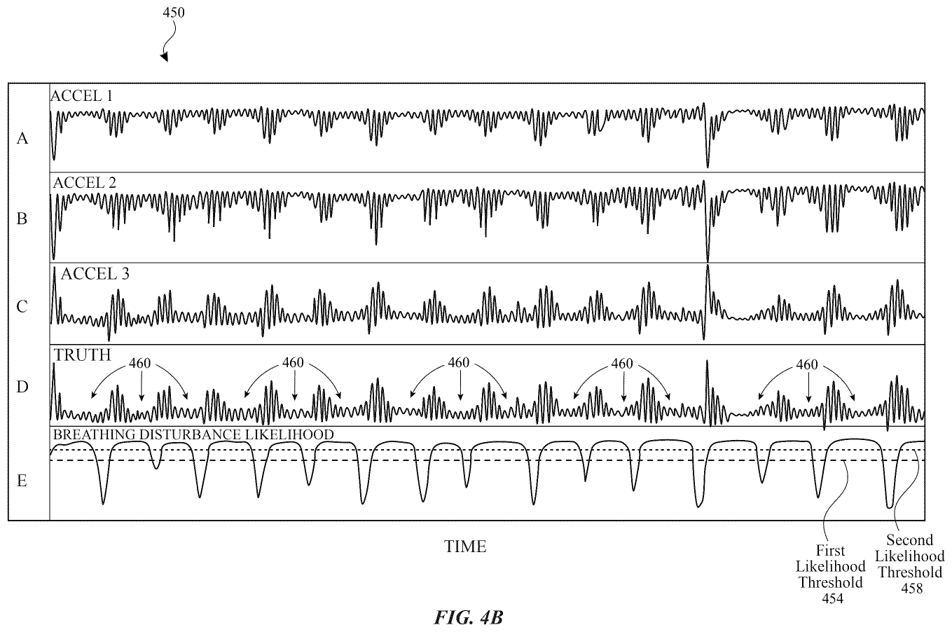

The data in the sleep tracking session 226 and/or in the breathing disturbance classification window 235 can be processed by the breathing disturbance classifier 210 as described in more detail with respect to FIGS. 4A-4B. In some examples, the breathing disturbance classification by breathing disturbance classifier 210 can begin in response to the end of the session (or a threshold period of time after the session or in response to a user request). In some examples, the breathing disturbance classification by breathing disturbance 210 can begin only after the confidence in the session is satisfied as determined by a quality check classifier (not shown in FIGS. 2A-2C, but described in more detail with respect to FIG. 6) between data collection and rest/active classification (between TO and T4) and the subsequent operation by breathing disturbance classifier 210. In some examples, the breathing disturbance classification by breathing disturbance classifier 210 can begin (e.g., upon the end of the session at T3 or T4), but can be aborted if ongoing, when the confidence in the session is not satisfied as determined by the quality check classifier. In some examples, the breathing disturbance classification determining breathing disturbances can be stored in memory and/or displayed to the user. For example, breathing disturbance classification determining breathing disturbances can be displayed or stored as a count of breathing disturbances during the session, a rate of breathing disturbances (e.g., breathing disturbances per hour), or another representation of breathing disturbances (e.g., a sequence of breathing disturbance intervals (e.g., consecutive periods of time classified as a likelihood of a breathing disturbance above a likelihood threshold)).

Although as described above the rest classifier 205A runs for a period (e.g., from T0 to T1) and the active classifier 205B runs for a period (e.g., starting at T2, and until T3), in some examples, the rest/active classifier can run for longer durations. For example, the rest/active classifier can run continuously (e.g., 24 hours a day, optionally only while the wearable device is on-wrist and/or not charging) or the rest/active classifier can run continuously between the user-defined bedtime and wake-up (or a threshold time before and/or after the user-define bedtime/wake-up), and multiple sleep tracking sessions and breathing disturbance classification windows can be identified (rather than the one breathing disturbance classification window illustrated in FIG. 2C). The samples from each identified breathing disturbance classification window can be processed to identify breathing disturbances, as described herein. In some examples, rather than operating continuously, the operation of the rest/active classifier can be periodic, intermittent or in response to one or more triggers.

In some examples, the breathing disturbance classification determining breathing disturbances can be displayed and/or stored only when confidence in the session is satisfied as indicated by the quality check classifier (not shown in FIGS. 2A-2C, but described in more detail with respect to FIG. 6). The quality check by the quality check classifier can begin in response to the end of the session. In some examples, the quality check classifier can estimate whether the motion data collected by the wearable device corresponds to the wearable device remaining on-wrist during the session (e.g., between the indication of on-wrist by an optical sensor). Using motion data can save power and reduce light while a user is sleeping as compared with using the optical sensor for on-wrist detection during the sleep tracking session. In some examples, one or more temperature sensors of the wearable device can be used to determine whether wearable device is on-wrist.

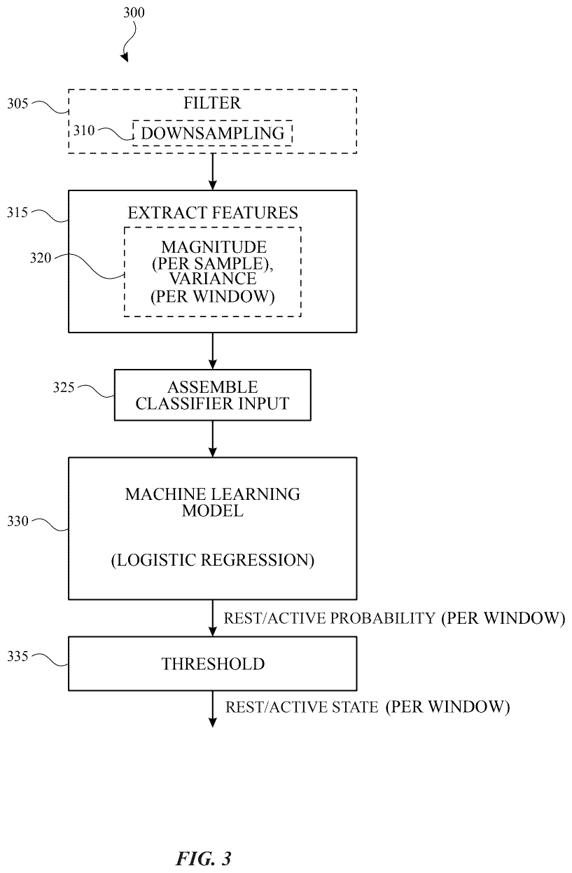

FIG. 3 illustrates an example process for a rest/active classifier according to examples of the disclosure. Process 300 can be performed by processing circuitry including one or more processors 108 and/or DSP 109. Process 300 can be performed in real-time (e.g., as sufficient data for processing is received) once the rest/active classification is triggered (e.g., in accordance with satisfying one or more first/second triggering criteria). It is understood that process 300 is an example process and alternative processes can be used to determine a rest or active state.

At 305, the rest/active classifier can optionally filter the data input into the classifier. The data can include motion data from a three-axis accelerometer (or other suitable motion and/or orientation sensor). In some examples, the filtering can be a low-pass filter to filter out high frequency noise (e.g., outside of the frequency of expected user motion). In some examples, the motion data can also be down-sampled at 310. For example, the accelerometer may capture motion data at a first sampling rate (e.g., 60 Hz, 100 Hz, 125 Hz, 250 Hz, etc.) and the motion data can be down-sampled (e.g., using multi-stage polyphase filter) to a lower rate (e.g., 4 Hz, 8 Hz, 10 Hz, 30 Hz, 50 Hz, etc.). Down-sampling the motion data can reduce the number of samples and thereby reduce the processing complexity. In some examples, the motion data can be processed without down-sampling and/or without filtering.

At 315, the rest/active classifier can extract one or more features from the motion data. In some examples, the one or more features can be extracted for samples in each “rest/active classifier window” or simply “window” in the context of rest/active classification (e.g., distinct from an “epoch” which can be a longer duration window for breathing disturbance classification). For example, the motion data be divided into N non-overlapping windows that include M samples of acceleration in each dimension (X, Y, Z) of a three-channel accelerometer. In some examples, the window can be between 1-60 seconds in duration. In some examples, the window can be between 1-30 seconds in duration. In some examples, the window can be between 1-10 seconds in duration. In some examples, the window can be between 2-5 seconds.

In some examples, the one or more features can include a magnitude feature for each sample in the window and a variance feature for the samples in the window (e.g., at 320). The magnitude of each of the M samples in a window can be computed using equation (1):

$$magnitude = \sqrt{{X}^{2} + {Y}^{2} + {Z}^{2}} \qquad ( 1 )$$

where X, Y and Z represent the x-axis accelerometer measurement for a sample, y-axis accelerometer measurement for a sample, and z-axis accelerometer measurement for a sample, respectively. The variance of the M magnitude values for the window can be computed using equation (2):

$${σ}^{2} = \frac{{\sum }_{i = 1}^{M} {( {mag}_{i} - \overline{mag} )}^{2}}{M} \qquad ( 2 )$$

where σ² represents the variance for the window, M represents the number of samples in the window, magi represents the magnitude of the i^{th} sample, and m̅a̅g̅ represents the mean magnitude for the window.

At 325, the input for the classifier can be assembled. The rest/active classifier input can be assembled from features for N windows and thus the input can correspond to a longer duration period than the window used for extraction of the magnitude and variance features described above (e.g., corresponding to periods of 30 seconds, 60 seconds, 90 seconds, 120 seconds, etc.). In some examples, the input can include N*(M+1) features. In some examples, the input can be compressed to reduce the number of features. For example, the features from multiple windows can be reduced by sum-pooling the features for k consecutive windows to reduce the input to

$$\frac{N}{k} * ( M + 1 )$$

features. In some examples, k can be between 2-10. In some examples, k can be between 3-8. A buffer can be used to store data (raw acceleration data and/or extracted magnitude and variance features) corresponding to the longer duration period such that sufficient data can be available as input to the rest/active classifier.

At 330, the classifier input can be processed with a machine-learning (ML) model, such as a logistic regression. It is understood that logistic regression is just one example of an ML model and other models can be used such as gradient-boosted trees, random forests, neural networks, support vector machines, etc. The output of the ML model can be a confidence value representing the likelihood such as a probability (between 0 and 1) that the user is in a resting state. In some examples, the ML model can output a confidence value for each period of time corresponding to the duration of the window (e.g., using a sliding window on the data buffer). For example, a first input of N windows (e.g., windows 1-100) can be used to calculate a first confidence value, a second input of N windows (e.g., windows 2-101) can be used to calculate a second confidence value, and so on. Thus, the output of the ML model can be represented as an array of confidence values (per window).

At 335, a threshold can be applied to the output of the ML model to detect a rest or an active state, with different parameters used for rest classification than for active classification. For example, a rest state (e.g., at T1 for the rest classification beginning at T0 in FIGS. 2B-2C) can be detected when the rest confidence value is greater than a first threshold confidence value for a first threshold number of windows in a given first period. For example, the rest state can be detected when the rest state confidence is greater than the first threshold confidence value (e.g., 85%, 90%, 95%, etc.) for most or all of (e.g., for 95%, 100%, etc. of) a first period (e.g., of a duration of 3 minutes, 5, minutes, 10 minutes, etc.). An active state (e.g., at T3 for the active classification beginning at T2 in FIGS. 2B-2C) can be detected when the rest confidence value is less than a second threshold confidence value for a second threshold number of windows in a given second period. For example, the active state can be detected when the rest state confidence is less than the second threshold confidence value (e.g., 70%, 75%, 80%, etc.) for (e.g., for 10%, 15%, etc. of) a second period (e.g., of a duration of 15 minutes, 20, minutes, 30 minutes, etc.). In some examples, the first threshold confidence value and the second threshold confidence value can be the same. In some examples, the first threshold confidence value and the second threshold confidence value can be different such that it may require a relatively higher confidence of rest to enter the rest state (from the non-resting/active state) and a relatively lower confidence of rest to enter the active state (from the non-active/rest state). In some examples, detecting the rest state can require the first threshold number of windows in the first period be consecutive (e.g., a threshold number of consecutive minutes with a rest state confidence above the threshold), whereas detecting the active state may not require the second threshold number of windows in the second period be consecutive (e.g., a threshold number of consecutive or non-consecutive minutes of activity within a longer period).

In some examples, the output of process 300 is a start time (e.g., corresponding to when transition from non-rest to rest state occurs at T1 of FIGS. 2B-3C) and an end time (e.g., corresponding to when transition from non-active to active state occurs at T3 of FIGS. 2B-3C).

FIG. 4A illustrates an example block diagram of breathing disturbance tracking including feature extraction and mask generation for breathing disturbance classification according to examples of the disclosure. Breathing disturbance classifier 410 can correspond to breathing disturbance classifier 210. Block diagram 400 illustrates input motion data 402A from a three-axis accelerometer (e.g., a three-channel motion sensor) that can be taken from a raw data buffer and/or from the output of ADC 105a. The input motion data can be down-sampled (e.g., from frequency f1 to frequency f2, less than frequency f1) and/or low-pass filtered in a down-sampling block 403A (e.g., implemented in a hardware or software). Additionally or alternatively input motion data can be further down-sampled and/or filtered in down-sampling block 403B (e.g., from frequency f2 to frequency f3, less than frequency f2). Additionally or alternatively input motion data can be down-sampled and/or low-pass filtered in rest/active classifier 405 (e.g., corresponding to rest/active classifier 205 in FIG. 2A, optionally implemented with a separate rest classifier and a separate active classifier.).

The extraction of features from the motion data can be performed from different streams of the motion data. For example, block diagram 400 includes multiple feature extraction blocks including rest/active classifier 405 (e.g., with feature extraction described with respect to FIG. 3), feature extraction block 406, and feature extraction and first mask generator block 422. Each of these blocks receives motion data, but optionally, different streams of motion data. For example, as shown in FIG. 4A, motion data 402A at frequency f1 is input to rest/active classifier 405, motion data 402B at frequency f2 is input to feature extraction and first mask generator block 422, and motion data 402C at frequency f3 is input to feature extraction block 406. Although three different sampling frequencies are shown for the motion data, it is understood that one sampling frequency or a different number of different sampling frequencies can be employed. Additionally or alternatively, different filtering can be applied to the motion data. For example, the motion data 402C can be low-pass filtered (e.g., via down-sampling block 403B), and the motion data 402B can be high-pass filtered and/or bandpass filtered for extraction of motion and/or respiration features at feature extraction and first mask generator block 422. For example, the one or more motion features can be extracted by the feature extraction and first mask generator block 422 from a 3-axis stream of the motion data corresponding to motion data 402B, further filtered by a high-pass filter and/or from a 3-axis stream of the motion data corresponding to motion data 402B without the further high-pass filtering. The one or more time-domain respiration features can be extracted by feature extraction and first mask generator block 422 from one selected axis (or more than one) of a 3-axis stream of the motion data further filtered using a band-pass filter. The one or more frequency domain features can be extracted by the feature extraction block 406 from the 3-axis stream of the motion data corresponding to motion data 402C without the further high-pass filtering. The selection of the one axis of the 3-axis stream can be performed using the 3-axis stream of the motion data without the further high-pass filtering. In some examples, high-pass filter can filter out some or all of the respiration band (e.g., filter out data below a threshold frequency, such as 0.5 Hz), and band-pass filter can filter out some or all data outside the respiration band (e.g., passing data between in a range of frequencies, such as between 0.1 Hz and 0.6 Hz).

The motion data can be divided into epochs for feature extraction (e.g., by an epoching block implemented in hardware or software, not shown). In some examples, the epoching can be achieved using a sliding window of the duration of an epoch (e.g., accessing motion data from a data buffer corresponding to the epoch duration). The epoching can be performed on multiple streams of the accelerometer data corresponding to motion data 402A-402C. In some examples, the same epochs are used for different forms of feature extraction. In some examples, different epochs can be used for different forms of feature extraction. For example, feature extraction at feature extraction block 406 can use a first epochs, feature extraction at “feature extraction and first mask generator” 422 can use second epochs (e.g., different than the first epochs), and/or feature extraction at rest/active classifier 405 can use third epochs (e.g., different than the first and second epochs). In some examples, the sliding window applied for epoching and/or feature extraction can be the same. In some examples, the sliding window applied for epoching and/or feature extraction can be different. For example, the feature extraction at rest/active classifier 405 can use a different sliding window than the feature extraction at feature extraction block 406 and feature extraction and first mask generator block 422. Using the same sliding window, however, can allow for various blocks to generate outputs at a consistent cadence. In some examples, the sliding window can be 0.5 seconds, 1 second, 2 seconds, 5 seconds, 10 seconds, 30 seconds, etc. In some examples, the epochs can be 10 seconds, 20 seconds, 30 seconds, 45 seconds, 60 seconds, etc.

As described in more detail herein, some extracted features are used as input for a model for classifying pluralities of likelihoods of the input including one or more physiological events. For example, FIG. 4A illustrate a machine learning (ML) model 408 that is trained to accept the output of feature extraction block 406 and to output a pluralities of likelihoods of the input including one or more physiological events. For example, training can be based on sets of motion data from one or more motion sensors, such as the one or more accelerometer streams illustrated in plots A-C of plots 450 in FIG. 4B and a “ground truth” provided by one or more additional sensors (e.g., electroencephalogram sensors, airflow sensors, respiratory effort sensors, pulse oximetry sensors, etc.) indicating breathing disturbances 460 (and their durations) as shown in plot D of plots 450 in FIG. 4B. ML model 408 can be trained to output likelihoods of a breathing disturbance corresponding to the epochs according to a sliding window, as shown in plot E of plots 450 in FIG. 4B. As described in more detail herein, one or more threshold, such as first likelihood threshold 454 and/or second likelihood threshold 458 can be used for mask generation and/or breathing disturbance counting.

In some examples, ML model 408 outputs a likelihood at a cadence of the sliding window that an epoch corresponding to the sliding window includes a physiological event such as a breathing disturbance associated with an apneic event. In some examples, the likelihood is represented as a probability between 0 and 1, where 0 represents a minimum probability (e.g., no breathing disturbance) and 1 represents a maximum probability (e.g., breathing disturbance). It is understood that the likelihood can be represented in other quantitative or qualitative ways (e.g., binary state based on the first likelihood threshold 454 (e.g., higher likelihood of breathing disturbance above the threshold), low, medium, high states based on multiple thresholds such as first likelihood threshold 454 and second likelihood threshold 458 (high likelihood of breathing disturbance above both thresholds, medium likelihood of breathing disturbance between both thresholds, low likelihood of breathing disturbance below both thresholds), etc.). The output of the ML model 408 can be used for mask generation (e.g., second mask generator 428) and/or as an input to an event counting block 412. As described in more detail herein, the output of ML model 408 (pluralities of likelihoods) can be masked by one or more masks (e.g., first mask, second mask, third mask), and then processed at the event counting block 412 to determine one or more breathing disturbances.

The ML model 408 can be implemented by hardware, software, firmware or a combination thereof. In some examples, the ML model includes one or more of the following characteristics: a supervised learning model, a semi-supervised learning model, an unsupervised learning model, a reinforcement learning model, a deep learning model. In some examples, the ML model includes artificial intelligence or uses one or more neural networks such as artificial neural networks (ANNs), feed forward neural networks (FFNNs), recurrent neural networks (RNNs), gated recurrent units (GRUs), Long Short Term Memory (LSTM), convolutional neural network (CNNs). In some examples, ML model 408 includes one or more CNNs (e.g., to extract features from input motion data) followed in series by one or more RNNs (e.g., to predict a probability of apnea/hypopnea events occurring). In some examples, the ML model can include a logistic regression model, linear regression model, ridge regression model, decision trees, gradient-boosted trees, random forests, support vector machines, K-nearest neighbor (KNN) machines, etc.

As described in more detail herein, some extracted features are used to generate one or more data masks. For example, a first data mask can be generated by feature extraction and first mask generator block 422 based on the output of the rest/active classifier and the sliding window for feature extraction at feature extraction and first mask generator block 422. This first data mask can correspond to the first mask in FIG. 2C, corresponding to a rest (non-active) session. A second data mask can be generated by second mask generator 428. The second data mask can be generated based on one or more features (e.g., including at least one motion features) and the first data mask generated by feature extraction and first mask generator block 422. Additionally or alternatively, in some examples, the second data mask can be generated based on the pluralities of likelihoods output by ML model 408. Additionally or alternatively, in some examples, the second data mask is generated based on the third data mask generated by third mask generator 436. A third data mask can be generated by third mask generator 436. The third data mask can be generated based on one or more features (e.g., including one or more motion features and one or more respiration features).

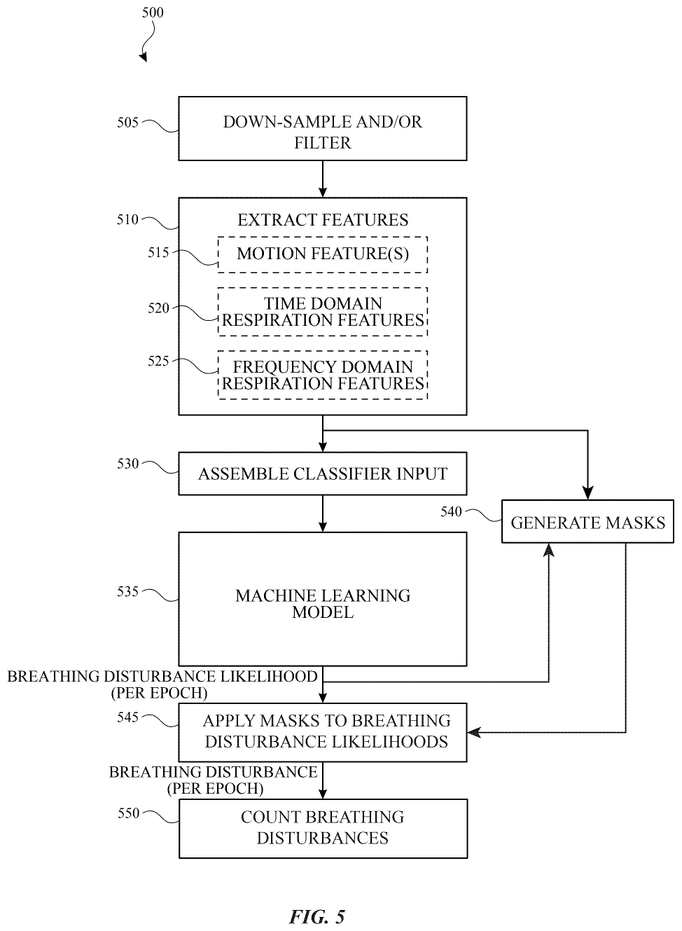

FIG. 5 illustrates an example process for a breathing disturbance classifier according to examples of the disclosure. Process 500 can be performed by processing circuitry including one or more processors 108 and/or one or more DSPs 109 (e.g., corresponding to breathing disturbance classifier 410). In some examples, process 500 can be performed partially in real-time (e.g., as sufficient data for processing is received), partially in a cadence during the session, and/or partially at the end of the session. In some examples, process 500 can be performed entirely at the end of the session (e.g., as shown in FIG. 2B).

At 505, the breathing disturbance classifier can optionally down-sample and/or filter the data input into the classifier. The data can include motion data from a three-axis accelerometer (or other suitable motion and/or orientation sensor). In some examples, the filtering can be a low-pass filter to filter out high frequency noise (e.g., outside of the frequency of expected user motion/respiration). In some examples, the motion data can be down-sampled at down-sampling block 403B (and/or down-sampling block 403A, when included in breathing disturbance classifier 410). For example, the accelerometer may capture motion data at a first sampling rate (e.g., 60 Hz, 100 Hz, 125 Hz, 250 Hz, etc.) and the motion data can be down-sampled (e.g., using on or more multi-stage polyphase filters) to a lower rate (e.g., 4 Hz, 8 Hz, 10 Hz, 30 Hz, 50 Hz, etc.). In some examples, down-sampling and low-pass filtering can be performed in real-time or in a cadence during the session to reduce the amount of data to be processed and/or stored. In some examples, the motion data can be processed without down-sampling and/or without low-pass filtering.

At 510, the breathing disturbance classifier can extract multiple features from the motion data. In some examples, the one or more features can include one or more motion features (e.g., at 515) (also referred to as movement features) (e.g., at feature extraction and first mask generator block 422), one or more time-domain respiration features (e.g., at 520) (e.g., at feature extraction and first mask generator block 422), and one or more frequency-domain respiration features (e.g., 525) (e.g., at feature extraction block 406). The multiple features can be computed for each epoch of motion data, though different epochs can be used for extraction of different features. The epoch can represent a window of motion data samples for the sleep session. In some examples, the epoch represents a window of motion data samples with a duration greater than the duration of the window used for rest/active classification (e.g., the rest/active classifier window). In some examples, the epoch can represent a window with a duration the same as the duration of the window used for rest/active classification. In some examples, the epoch can represent a window with a duration greater than the duration of the window used for rest/active classification. In some examples, the epoch can be between 10-120 seconds in duration. In some examples, the epoch can be between 20-60 seconds in duration. In some examples, the epoch can be between 30-90 seconds in duration. In some examples, the epoch can be between 45-60 seconds. In some examples, the feature extraction can be performed on epochs that define overlapping periods. For example, adjacent epochs can overlap by 5-60 seconds. In some examples, the overlap can be between 20-30 seconds. In some examples, the overlap can be between 1-5 seconds. Feature extraction is described in more detail below.

At 530, the input for the ML model (e.g., ML model 408) of the breathing disturbance classifier can be assembled. The input for the ML model of the breathing disturbance classifier can be assembled from features for N epochs and can correspond to a longer duration period (e.g., corresponding to 5 minutes, 10 minutes, etc.). In some examples, the input can include N*M features, where M features are extracted for each of N epochs. In some examples, the one or more frequency-domain respiration features (525 extracted at feature extraction block 406) can include one or more two-dimensional spectrogram representing the spectrum of frequencies of the motion data over time. To create each spectrogram (e.g., one for x-axis, one for y-axis, and one for z-axis), a one-dimensional motion signal divided into non-overlapping longer duration periods (e.g., again per axis), such as represented in the longer duration period in for the x-axis, y-axis, and z-axis in plots A-C of FIG. 4B. Each segment of the spectrogram is computed using the Fourier transform (e.g., fast Fourier transform (FFT), Short-Time Fourier Transform (STFT), etc.) for an epoch of the longer duration period. The result applied to each epoch using a sliding window for the longer duration period is a two-dimensional map in time-frequency domain, with a first (e.g., horizontal) axis representing time in seconds (e.g., from zero to the duration of the longer duration period), with a resolution of the sliding window duration (e.g., 2 seconds, 5 seconds, 10, seconds, 20 seconds, etc.), and with a second (e.g., vertical) axis representing frequency in Hz from a minimum to a maximum (e.g., from 0 Hz to 2 Hz). In some examples, the frequency resolution can be between 0.001 Hz and 0.1 Hz. In some examples, the frequency resolution can be between 0.01 Hz and 0.075 Hz. In some examples, the frequency resolution can be between 0.025 Hz and 0.05 Hz. It is understood that the resolution in time or frequency can be tuned to balance performance of the ML model and the volume of data to process and provide as input to ML model 408. The two-dimensional spectrograms for each axis of the motion data can be assembled as the input to ML model 408. A buffer can be used to store data (raw and/or filtered/down-sampled acceleration data and/or extracted features) corresponding to the longer duration period such that sufficient data can be available as input to the ML model of the breathing disturbance classifier.

At 535, the ML model input can be processed with an ML model. ML model 408 can be trained to output a likelihood of a breathing disturbance (e.g., an apnea/hypopnea probability) once per the sliding window duration. For example, for a 5 minute duration with a 10 second sliding window, the output of the ML model can include a 30-element vector of probabilities. For example, for a 10 minute duration with a 5 second sliding window, the output of the ML model can include a 120-element vector of probabilities. The output of the ML model 408 for each of the longer duration periods over the duration of the session can be concatenated. Each element of the vector can include a likelihood, such as a probability, computed with a cadence matching the sliding window. Thus, the output of the ML model 408 can be represented as a vector of breathing disturbance likelihood values (per epoch, at the sliding window cadence), such as shown in plot E in FIG. 4B.

At 540, one or more masks can be generated using one or more extracted features. At 545, one or more masks can be applied to the output of the ML model (e.g., pluralities of likelihoods of a breathing disturbance). As described herein, the one or more masks can include a first mask, a second mask, and/or a third mask. For example, the first mask can correspond to a rest (non-active) session (e.g., corresponding to the first mask in FIG. 2C), which can filter from the output of the ML model likelihoods of a breathing disturbance while the user is determined to be active or not resting/sleeping (e.g., periods in which a detected breathing disturbance is unrelated to an apneic condition). The first mask can be generated, as described with reference to FIG. 3, using a motion feature such a magnitude and/or variance features for the motion data. A second mask can be generated by second mask generator 428. The second mask can correspond to a transition from a pre-sleep state to a sleep state based on the first mask, which can further filter from the output of the ML model likelihoods of a breathing disturbance while the user is determined to be resting, but not yet sleeping (e.g., periods in which a detected breathing disturbance is unrelated to an apneic condition). The second mask can be generated based on one or more features (e.g., including at least one motion features, also referred to herein as movement features) and the first mask (e.g., generated by feature extraction and first mask generator block 422). Additionally or alternatively, in some examples, the second mask can be generated based on the pluralities of likelihoods output by ML model 408 and/or based on the third data mask generated by third mask generator 436. The first mask and the second mask can provide for counting breathing disturbances in the breathing disturbance classification window 235. A third mask can be generated by third mask generator 436, which can further filter from the output of the ML model corresponding to the breathing disturbance classification window 235 to remove one or more likelihoods that correspond to motion data failing to meet signal quality criteria (e.g., periods in which a detected breathing disturbance may be due to motion artifact or poor connection (e.g., poor contact) between the wearable device and the user unrelated to an apneic condition).

In some examples, the pluralities of likelihoods output by the ML model are converted to a binary output. For example, a likelihood threshold (e.g., first likelihood threshold 454) can be applied to the output vector from ML model 408 to generate a binary vector including “zeros” (e.g., for likelihoods below the likelihood threshold) and “ones” (e.g., for likelihoods above the likelihood threshold). In some examples, each mask is represented as a binary vector with “zeros” and “ones,” and applying the one or more masks includes vector multiplication with a binary vector representing the pluralities of likelihoods output by the ML model. In some examples, the vector multiplication can be achieved using AND gates or logical operations between each element of the binary vector representing the pluralities of likelihoods output by the ML model and each element of the respective mask or masks. In some examples, each mask nulls (e.g., multiplication by zero) or preserves (e.g., multiplication by unity) portions of the plurality of likelihoods of a breathing disturbance. For example, the first mask nulls active or non-resting periods, the second mask nulls quiet wake periods, and the third masks nulls remaining portions corresponding to poor signal quality (e.g., failing to satisfy one or more quality criteria). In some examples, the order of vector multiplication or application of AND gates or logical operations can be different in different implementations. For example, the first mask and the second mask can be combined (using multiplication, AND gates/logical operations) to generate an intermediate mask, which can be combined with the third mask to generate a final mask. The final mask can then be applied to the binary vector representing the pluralities of likelihoods output by the ML model. The order of mask operations can be varied (e.g., combine the second and third masks to generate the intermediate mask). As an alternative, each mask can be sequentially applied to the binary vector representing the pluralities of likelihoods output by the ML model. It is understood that other ordering can be used for application of masks. It is also understood that in some examples, fewer, more, or different masks can be applied.

At 550, one or more breathing disturbances during a session can be counted using the masked output of the ML model (e.g., pluralities of likelihoods of a breathing disturbance). For example, the counting can be performed using event counting block 412, which can be implemented as a processing algorithm or can implemented as a machine learning model to determine one or more breathing disturbances during a session. In some examples, the masked output of the ML model is a vector with elements for pluralities of likelihoods of a breathing disturbance remaining after masking. The counting can include identifying subsets of the pluralities of likelihoods that satisfy one or more first criteria for classification as a breathing disturbance. For example, a breathing disturbance can be classified when the likelihood of a breathing disturbance is above a threshold likelihood for a threshold period of time. In other words, the first criteria for classification as a breathing disturbance can include a criterion that is satisfied when the first subset of the pluralities of likelihoods is greater than a first likelihood threshold, and a criterion that is satisfied when the first subset corresponds to a consecutive period of time that is greater than a first time threshold. In some examples, the threshold period of time can be defined based on a clinical definition of a breathing disturbance for sleep apnea. In some examples, the threshold period of time is 10 seconds. In some examples, a threshold number of consecutive likelihoods can correspond to the period of time, where the threshold number can be computed based on the threshold period of time divided by the cadence of the ML output (e.g., which can correspond to the sliding window), rounded up to the nearest integer number. For example, if the threshold period of time is 10 seconds and the sliding window is 1 seconds, then the threshold number is 10; if the threshold period of time is 10 seconds and the sliding window is 2 seconds, then the threshold number is 5; if the threshold period of time is 10 seconds and the sliding window is 3 seconds, then the threshold number is 4. In some examples, the threshold likelihood corresponds to the first likelihood threshold 454. In some examples, the threshold likelihood is between 0.6-0.9. In some examples, the threshold likelihood is between 0.7-0.8. In some examples, the threshold likelihood is 0.7, 0.725, 0.75, 0.775, etc.

In some examples, the counting can include converting the masked pluralities of likelihoods to a binary output. For example, the above-described likelihood threshold can be applied to the output vector including the masked pluralities of likelihoods to output a binary vector including “zeros” (e.g., for likelihoods below the likelihood threshold) and “ones” (e.g., for likelihoods above the likelihood threshold). In some examples, the above-described likelihood threshold can be applied to the output vector before masking.

In some examples, the counting can include grouping continuous vector elements with a “one” value as a predicted breathing disturbance, and a total number (e.g., occurrences) of predicted breathing disturbances can be counted when the corresponding duration is at or above the threshold. In some examples, the output of counting is a total number of predicted breathing disturbances. In some examples, the output of counting is a rate of predicted breathing disturbances. For example, the total number of predicted breathing disturbances can be divided by a period of time. In some examples, the period of time is the breathing disturbance classification window 235. In some examples, the period of time is the total duration of data meeting quality criteria (e.g., the duration of “ones” in the third mask). In some examples, the period of time is the total duration of data meeting quality criteria (e.g., the duration of “ones” in the third mask) within the breathing disturbance classification window 235. In some examples, the rate of predicted breathing disturbances is represented in units of events per hour for a sleep session. A representation of a number of the one or more of the physiological events in the first session (e.g., the count of breathing disturbances as a total number or rate) can be stored in memory of an electronic device. In some examples, the representation of the number (e.g., occurrences) of the one or more of the physiological events in the first session can be displayed to the user. In some examples, the representation of the number of the one or more of the physiological events in the first session can be stored as data for an application (e.g., health application) and/or shared with a medical or health care contact, when authorized by the user.

Returning back to feature extraction, feature extraction and first mask generator block 422 can be used to extract the one or more motion features (e.g., at 515) and one or more respiration features including one or more time-domain respiration features (e.g., at 520) and optionally one or more frequency domain respiration features. In some examples, frequency domain respiration features are not extracted by feature extraction and first mask generator block 422. As described herein, in some examples, feature extraction and first mask generator block 422 extracts features from motion data 402B, and optionally applies bandpass filtering to the motion data stream. The one or more motion features (e.g., at 515) and one or more respiration features can be extracted with a cadence that matches a sliding window (e.g., every 1 second, 2 second, 5 second, 10 seconds, etc.). In some examples, the one or more features include a “median signal peak amplitude,” a “respiration rate” respiration feature (e.g., mean respiration rate, variance of respiration rate, etc.) and/or a “maximum variance” motion feature. The feature extraction and first mask generator block 422 can include additional or alternative features.

The one or more motion features extracted can include a “maximum variance” motion feature. The maximum variance can be computed from among the epoched 3-axis accelerometer stream corresponding to motion data 402A (e.g., optionally without high-pass filtering and/or band-pass filtering) or epoched 3-axis accelerometer stream corresponding to motion data 402B. The variance of the magnitude for samples in the epoch be computed for each channel of the epoched 3-axis accelerometer stream in a similar manner as described above in equation (2), but for the single-axis magnitude of each sample in the epoch. The maximum variance among the three variance values for the 3-channels of epoched 3-axis accelerometer stream (e.g., a first variance value for a first channel, a second variance value for the second channel and a third variance for the third channel) can represent the maximum variance feature. Additionally or alternatively, in some examples, a natural logarithm of the maximum variance feature may be used as a motion feature.

In some examples, the one or more motion features extracted can include a “mean variance” motion feature. The magnitude (2-norm) of motion for each sample in the epoched high-pass filtered 3-axis accelerometer stream can be computed in a 2-Norm magnitude block (e.g., in a similar manner as described in equation (1) as applied to the epoched high-pass filtered 3-axis accelerometer stream). In some examples, the magnitude can be computed for the high-pass filtered 3-axis accelerometer stream prior to epoching (e.g., on a sample-by-sample basis). The variance of the magnitude for each of the samples in the epoch be computed. The mean variance feature can be computed as the mean of the computed variances across all the samples in the epoch. The mean variance feature can correlate with a wake state. Although described as a mean variance motion feature, additionally or alternatively, the one or more features can include the median variance or the mode variance (e.g., taking a median or a mode of the variances across all the samples in the epoch).

The one or more motion features can include a “motion count” motion feature. The motion count feature can be a determination of the number of motion samples in the epoch with magnitude of motion above a threshold. The magnitude (2-norm) of motion for each sample in the epoched high-pass filtered 3-axis accelerometer stream can be computed in a 2-Norm magnitude block 512 (e.g., in a similar manner as described in equation (1) as applied to the epoched high-pass filtered 3-axis accelerometer stream). In some examples, the magnitude can be computed for the high-pass filtered 3-axis accelerometer stream prior to epoching (e.g., on a sample-by-sample basis). The motion count feature can be determined by counting the number of samples or the fraction/percentage of samples in the epoch whose 2-norm magnitude of motion is above a threshold. The motion count feature can indicate an amount of motion above some noise threshold for the epoch.

The one or more motion features can include a “motion integration” motion feature. The motion integration feature can sum the magnitudes for the sample in the epoch by integrating the magnitudes as scaled by a dx term (e.g., ∫ magnitude·dx), where dx can be the sampling period (inverse of the sampling rate after down-sampling). The magnitude (2-norm) of motion for each sample in the epoched high-pass filtered 3-axis accelerometer stream can be computed in a 2-Norm magnitude block as described above. The motion integration feature can indicate the overall magnitude of motion for the epoch. The motion integration feature can be useful for identifying slower, sustained movements in the epoch, whereas the motion count feature can be useful for identifying faster movements (e.g., higher frequency movements/transients).

The one or more motion features can include a “motion integration mean” motion feature. The motion integration mean feature can be a mean of the “motion integration” feature described above. The motion integration mean feature can indicate the average of the overall variability in the magnitude of motion for the epoch. The motion integration mean feature can be useful for potentially identifying short-term, high-motion segments, which may correspond to short wake bouts. Although described as a motion integration mean feature, additionally or alternatively, the one or more motion features can include the motion integration median or the motion integration maximum.

The above motion features are examples of one or more motion features that could be extracted. It is understood that addition, fewer, and/or different motion features can be extracted for use in breathing disturbance classification. In some examples, the breathing disturbance classification can use the “maximum variance” feature, the “motion count” feature, and the “motion integration” feature. In some examples, the breathing disturbance classification can further use the “mean variance” feature and the “motion integration mean” feature. In some examples, the breathing disturbance classification uses only the “maximum variance” feature among the motion features.

In some examples, frequency-domain respiration features are not extracted by feature extraction and first mask generator block 422. In some examples, one or more frequency respiration features are extracted by feature extraction and first mask generator block 422. For example, the one or more frequency-domain respiration features can include one or more measures of the variability in a motion-sensor-derived respiration signal. In some examples, the one or more features can be computed from one-axis of the epoched 3-axis accelerometer stream (e.g., without high-pass filtering or band-pass filtering). The one-axis of the epoched 3-axis accelerometer stream can be selected for each epoch by the best axis estimation block (not shown) as the axis with the best respiration signal (e.g., based on a signal-to-noise ratio (SNR)). A frequency domain representation can be computed for each axis of the epoched 3-axis accelerometer stream in order to determine a best respiration signal. For example, a Fourier transform (e.g., fast Fourier transform (FFT)) can be computed for each axis and/or a power spectral density (PSD) can be computed for each axis. In some examples, the mean can optionally be subtracted from the epoched 3-axis accelerometer stream before computing the frequency domain representation (e.g., de-trending). An SNR can be computed for each axis of the 3-axis accelerometer stream based on the frequency representation. The “signal” of the SNR can be estimated by identifying a maximum peak in the frequency representation and computing spectral power (absolute value squared of the FFT) within a frequency-domain window around the maximum peak (e.g., within a range of a fundamental frequency). In some examples, a folded spectrum can be computed by summing the power over one or more harmonics of the frequency-domain window (e.g., optionally including some of the side-lobe bins around the fundamental frequency), and the spectral power can be computed based on the largest peak in the folded spectrum (e.g., the dominant frequency across multiple harmonics) and summing the power over the multiple harmonics including the side-lobe bins of the dominant frequency. In some examples, the “noise” of the SNR can be estimated by computing the spectral power outside the frequency-domain window around the maximum peak. The SNR can be computed from the ratio of the above defined signal and noise. The axis with the best respiration signal can be selected for an epoch based on the axis with the maximum SNR among the three axes for the epoch.

It should be understood that the above description of determining the SNR is an example, and the SNR can be computed in other ways and/or the axis with the best respiration signal can be determined in other ways. For example, the SNR can be computed, in some examples, as the log of the ratio of the “signal” described above to the total power of the spectrum (without computing the noise as described above). In some examples, rather than computing the best axis, the respiration signal can be extracted using singular spectrum analysis (SSA), principal component analysis (PCA), or rotation angles (RA). However, the above SNR approach can reduce processing complexity relative to SSA, PCA and RA, while providing the desired performance for breathing disturbance classification.

In some examples, the frequency-domain respiration features can include one or more “spectral power” respiration features for the selected best axis for one or more frequency ranges. The power spectral density (PSD) can be computed from the epoched 3-axis accelerometer stream (e.g., using an FFT block), optionally after de-trending. The spectral power feature can be a relative spectral density computed by the expression:

$$\frac{band power}{total power} ,$$

where the band power can be computed by integrating the PSD within the frequency limits of the band and the total power can be computed by integrating the total PSD. In some examples, the extraction of frequency-domain respiration features can include computing a first relative spectral power in the frequency range (e.g., 0.01-0.04 Hz), a second relative spectral power in the frequency range (e.g., 0.04-0.1 Hz), a third relative spectral power in the frequency range (e.g., 0.1-0.4 Hz), and a fourth relative spectral power in the frequency range (e.g., 0.4-0.9 Hz). The relative spectral density features can be useful because heart rate and/or respiration rate can have different modulations of power in these different frequency bands for a sleep state as compared with an awake state.

In some examples, the frequency-domain respiration features can include a “spectral entropy” respiration feature. The spectral entropy feature can be calculated from the selected best axis (optionally after de-trending). For example, the PSD can be calculated from an FFT, and the spectral entropy can be calculated from the PSD. For example, the spectral entropy can be calculated by normalizing the PSD (e.g., to sum to 1), treating the normalized PSD as a probability density function (PDF), and computing the Shannon Entropy. The spectral entropy can be useful for sleep/wake classification because a more regular breathing pattern associated with sleep can include a sharper PSD and therefore a lower spectral entropy.

In some examples, the frequency-domain respiration features can include a “respiration rate” respiration feature. The respiration rate feature can be calculated from the selected best axis (optionally after de-trending). In some examples, the frequency domain representation of the best axis can be computed using an FFT, and a frequency with the highest peak in the spectral output of the FFT can be identified as the respiration rate. Calculating the respiration rate in frequency domain can optionally provide for a more robust measurement (e.g., less susceptible to noise) compared with the time domain “respiration rate” respiration feature. In some examples, the respiration rate can be converted to a number-of-breaths per period of time (e.g., per minute). The respiration rate can be useful to identify sleep state due to an understanding of how respiration rate changes in different stages of sleep.

The above frequency-domain respiration features are examples of one or more frequency-domain respiration features that could be extracted. It is understood that addition, fewer, and/or different frequency-domain respiration features can be extracted for use in breathing disturbance classification. In some examples, the sleep/wake classification can use the “spectral power” feature and the “spectral entropy” feature. In some examples, the breathing disturbance classification can further use the “respiration rate” feature.

One or more Time-domain respiration features can be extracted by feature extraction and first mask generator block 422. Extracting time-domain respiration features can be based on identifying peak and valley indices in the epoched band-pass filtered 3-axis accelerometer stream and time intervals between peaks and valleys. The peaks and valleys can be associated with inhales and exhales (with the amplitude associated with breath intensity), and the time intervals between the peaks and valleys can be associated with breath times and durations. In some examples, these quantities can be extracted for the epoch, and the most stable quantities among these can be used for subsequent time-domain feature extraction, as described in more detail below.

In some examples, the one or more time-domain respiration features can be computed from one-axis of the epoched, band-pass filtered 3-axis accelerometer stream, where the one axis is selected is accordance with the operation of best axis estimation block.

Because the time-domain respiration features are optionally extracted from motion data (e.g., one selected axis of the epoched 3-axis accelerometer stream), the respiration signal can be susceptible to motion artifacts (e.g., motion unrelated to respiration). In some examples, the presence of a motion artifact can be estimated by motion artifact detection block (not shown) using the 3-axis accelerometer stream output of a band-pass filter. A motion artifact detection block can compute a maximum absolute variance across the 3-axis band-pass filtered accelerometer stream in a similar manner to maximum variance motion feature described above. However, rather than computing one maximum variance for an epoch as described for the maximum variance motion feature, the maximum absolute variance computed by motion artifact detection block can using a smaller sliding window. In some examples, the sliding window can be between 1-10 seconds in duration. In some examples, the sliding window can be between 2-5 seconds in duration. In some examples, the sliding window can have the same duration as the rest/active classifier window. After computing the maximum absolute variance using the sliding window, the maximum absolute variances for multiple windows can be thresholded. For example, the motion artifact detection block can output an array of binary values (a binary array) with a binary output value indicative of a motion artifact for the window when the maximum absolute variance is above a threshold (e.g., “one”) and a binary output indicative of a no motion artifact for the window when the maximum absolute variance is below the threshold (e.g., “zero”). The output of the motion artifact detection block can be sampled at the same rate as the output of down-sampling and/or filtering block 504 (though the maximum absolute variances were determined on a per-window basis with each window including multiple samples). In some examples, to mitigate the effect of filter transients, the samples indicative of a motion artifact in the binary array can be “padded” such that a threshold number (e.g., 2, 3, 5, 8, 10, etc.) of samples on either side of a sample indicative of a motion artifact can also be marked as indicative of a motion artifact (even though the maximum absolute variance of the sample may be below the threshold).

As described herein, time domain respiration features can be based on peaks and valleys detected optionally in the selected axis of the epoched 3-axis accelerometer stream. The samples in an epoch that are not masked out as including a motion artifact (which are filtered out) can be processed to identify peak and valley locations with amplitudes (absolute value) above a threshold. In some examples, the threshold can be determined on a per-epoch basis by computing the standard deviation of the selected axis of the epoched 3-axis accelerometer stream and multiplying the standard deviation by a scaling parameter. In some examples, the scaling parameter can be 1. In some examples, the scaling parameter can be greater than one or less than 1.

After computing the peaks and valleys (filtered for motion artifacts), inter-breath intervals (IBIs) can be computed by taking time differences between adjacent peak timestamps (inter-peak intervals) and/or the time difference between adjacent valley timestamps (inter-valley intervals). The IBIs can be indexed for storage using the interval start timestamps (e.g., peak start timestamps or valley start timestamps).

The identified peaks and valleys, as well as the inter-peak intervals and inter-valley intervals, can be filtered to remove portions from samples of the epoch that are contaminated by motion artifacts. For example, a peak that overlaps at least partially with samples contaminated by motion artifacts, a valley that overlaps at least partially with samples contaminated by motion artifacts, or an IBI that overlaps with motion artifacts can filtered out (e.g., to ensure that both the start point and end point of each breathing interval is free from motion artifacts). For example, a peak or valley may be detected at or near samples contaminated with motion artifacts can be masked out and/or breath intervals contaminated with motion artifacts can be masked out.

For the feature extraction, either the peaks (and inter-peak intervals) or the valleys (and inter-valley intervals) can be selected based on which show less variability. In some examples, the variability can be determined based on a standard deviation or a median absolute derivation of the IBIs within each epoch. For example, peaks (and inter-peak intervals) can be used if the variability for inter-peak intervals is lower than the variability for inter-valley intervals for the epoch, or the valleys (and inter-valley intervals) can be used if the variability for inter-valley intervals is lower than the variability for inter-peak intervals.

The one or more time-domain respiration features can include a “number of breaths” respiration feature indicating a number-of-breaths detected for the epoch, which can be determined by counting the number of peaks or valleys after the peak/valley and IBI detection and motion artifact filtering described above. The one or more time-domain respiration features can include a “respiratory amplitude variability” respiration feature for the epoch. The respiratory amplitude variability feature can be computed by computing the standard deviation of the amplitude of the peaks (or valleys) and normalizing the standard deviation of the amplitude of the peaks (or valleys) by the mean of the amplitude of the peaks (or valleys). In some examples, the one or more time-domain respiration features can include a “respiratory amplitude median” respiration feature for the epoch. The respiratory amplitude median feature can be computed by computing the median of the amplitude of the peaks (or valleys). In some examples, the one or more time-domain respiration features can include a respiratory amplitude mean (e.g., mean of the amplitude of the peaks (or valleys)) and/or a respiratory amplitude mode (e.g., mode of the amplitude of the peaks (or valleys)).

The one or more time-domain respiration features can include one or more respiratory rate variability (breath-to-breath variability) features for the epoch. A first respiratory rate variability feature can be a “mean-normalized median absolute deviation” respiration feature. This first respiratory rate variability feature can be computed by taking the difference between the instantaneous IBI and the median IBI for the epoch, and then normalizing by the mean IBI for the epoch. A second respiratory rate variability feature can be a “mean-normalized range” respiration feature. This second respiratory rate variability feature can be computed by taking the difference between the maximum and minimum IBI values for the epoch, and then normalizing by the mean IBI for the epoch. A third respiratory rate variability feature can be a “standard deviation” respiration feature. This third respiratory rate variability feature can be computed by taking the standard deviation of the IBI values for the epoch. A fourth respiratory rate variability feature can be a “root mean squared of successive differences” respiration feature. This fourth respiratory rate variability feature can be computed by taking the root-mean-squared deviations between successive peaks (or valleys) for the epoch.

Due to the motion artifact filtering or due to no breaths being detected in the epoch, in some examples, there may be insufficient data to compute one or more of the time-domain respiration features (e.g., except for the number of breaths feature, which is zero in such a case). For such epochs, the features can be assigned with predetermined values that correspond to a relatively high likelihood of a wake state (e.g., based on the empirical data). In some examples, predetermined values can be a percentile (e.g., 75th percentile, 85th percentile, 95th percentile) for each feature in the empirical data for a person who is awake.

The above time-domain respiration features are examples of one or more time-domain respiration features that could be extracted. It is understood that addition, fewer, and/or different time-domain respiration features can be extracted for use in breathing disturbance classification. In some examples, the breathing disturbance classification can use the “number of breaths” feature, the “respiratory amplitude variability” feature, the “mean-normalized median absolute deviation” feature, the “mean-normalized range” feature, the “standard deviation” feature. In some examples, the breathing disturbance classification can further use the “root mean square of successive differences” feature and the “respiration amplitude median” feature.

Additionally, feature extraction and first mask generator block 422 can output the first mask, which can be a binary vector at the cadence of the sliding window representing rest or active states (e.g., with “ones” representing rest state and “0” representing active state). In some examples, generating the first mask includes taking the output of rest/active classifier 405 and converting the output to the cadence of the sliding window used for the likelihoods output by ML model 408. In some examples, feature extraction and first mask generator block 422 can implement thresholding described at block 335 when the output of the active/rest classifier is not binary.

Returning back to the generation of the third mask, third mask generator 436 can generate the third mask based to identify and filter out segments of the motion data with insufficient quality (e.g., due to motion artifacts and/or poor contact or off-wrist conditions). The third mask can have the same cadence to match the sliding window for the other mask generators and ML model 408. The third mask can be generated based on one or more features from the feature extraction and first mask generator block 422. The one or more features can include one or more motion features and one or more respiration features. In some examples, the one or more features include the maximum variance motion feature. In some examples, the one or more features include the median peak signal amplitudes. In some examples, the third mask can be a binary vector at the cadence of the sliding window representing signal quality states (e.g., with “ones” representing “valid or sufficient signal quality” state and “0” representing “invalid or insufficient signal quality” state).

In some examples, although not shown in FIG. 4A, the third mask generator 436 can receive a representation of the likelihood that the wearable device is on-wrist (e.g., from an optical sensor or other sensor). The above-described signal quality states can be multiplied (e.g., logical AND) with the binary on-wrist state (e.g., off-wrist represented as “zeros” and on-wrist represented as “ones”). In some examples, the third mask also includes an indication of whether the signal quality determination is inside or outside of the rest/non-active session (e.g., “zeros” for out of session and “ones” for in session). In some examples, this indication is multiplied (e.g., logical AND) to limit the third mask to in-session times to allow the combination of masks (e.g., easier when they are of the same length).

In some examples, a quality check classifier is optionally included to establish a signal quality in the breathing disturbance classification. In particular, a quality check classifier can evaluate one or more extracted features to provide a confidence in the motion data (e.g., indicative that the wearable device was worn by the user during the sleep tracking session 226 and/or during the breathing disturbance classification window 235). In some examples, the quality check classifier can use a subset of the multiple features used for mask generation and/or breathing disturbance classification. In some examples, the quality check classifier can use one or more extracted motion features, one or more time-domain respiration features, and/or one or more frequency-domain respiration features.

FIG. 6 illustrates an example process for a quality check classifier according to examples of the disclosure. Process 600 can be performed by processing circuitry including one or more processors 108 and/or DSP 109. In some examples, process 600 can be performed at the end of the session, or before, after, or in parallel with the breathing disturbance classification of process 500. In some examples, the subset of features can include the motion integration feature and the maximum variance motion feature. In some examples, the subset of features can include the spectral entropy feature and one (or more) of the relative spectral power features. In some examples, the subset of features can include a number-of-breaths per epoch feature. Using a subset of extracted features may be useful for reducing the size of the classifier input and therefore the complexity of the quality check classifier. Additionally, using extracted features from other mask generation and/or breathing disturbance classification can avoid the need to extract additional features. In some examples, the same features extracted for mask generation and/or breathing disturbance classification may be used for the quality check classifier.

At 605, the input for the quality check classifier can be assembled. The quality check classifier input can be assembled from a subset of extracted features for the multiple epochs of the sleep session (or optionally of the rest/non-active session or of the breathing disturbance classification window). In some examples, the subset of extracted features for all epochs of the session can be used for quality check classification. In some examples, the input can be compressed to reduce the number of features. For example, the features from multiple epochs can be reduced by sum-pooling the features for k consecutive epochs to reduce the input by a factor of 1/k.

At 610, the classifier input can be processed with a ML model, such as a logistic regression. It is understood that logistic regression is just one example of an ML model and other models can be used such as gradient-boosted trees, random forests, neural networks, support vector machines, etc. The output of the ML model can be a confidence value representing the likelihood (e.g., a probability between 0 and 1) that the motion data is of a quality that it can pass the quality check (thereby expressing confidence in the breathing disturbance classification based on the motion data). This quality check confidence value can correspond to the likelihood that the wearable device remained on-wrist (e.g., was not removed and resting on a table or other surface during the sleep session and/or during sub-sections of the sleep session) during the breathing disturbance classification window.

At 615, a threshold can be applied to the output of the ML model to detect a quality check result or state. For example, the quality check can be passed (passed state, e.g., represented by “ones” in a binary vector) when the quality confidence value is greater than a threshold confidence value, and the quality check can be failed (failed state, e.g., represented by “zeros” in a binary vector) when the quality confidence value is less than the threshold. As described herein, failing the quality check can result in masking some results and/or forgoing published the breathing disturbance tracking results to the user (and/or discarding the breathing disturbance tracking results), whereas passing the quality check can result in storing and/or publishing the breathing disturbance tracking results.

Returning back to the generation of the second mask, second mask generator 428 can generate the second mask based to identify and filter out segments of the motion data corresponding to rest without sleep (e.g., quiet wake). The second mask can have the same cadence to match the sliding window for the other mask generators and ML model 408. The second mask can be generated based on the first mask and one or more features from the feature extraction and first mask generator block 422, and/or the output of ML model 408 (e.g., the pluralities of likelihoods of a breathing disturbance). The one or more features can include one or more motion features and/or one or more respiration features. In some examples, the one or more features include the maximum variance motion feature. In some examples, the second mask can be a binary vector at the cadence of the sliding window representing signal quality states (e.g., with “ones” representing “an ‘in-bed’ or sleep state” state and “zeros” representing “an ‘out-of-bed’ or non-sleep resting” state).

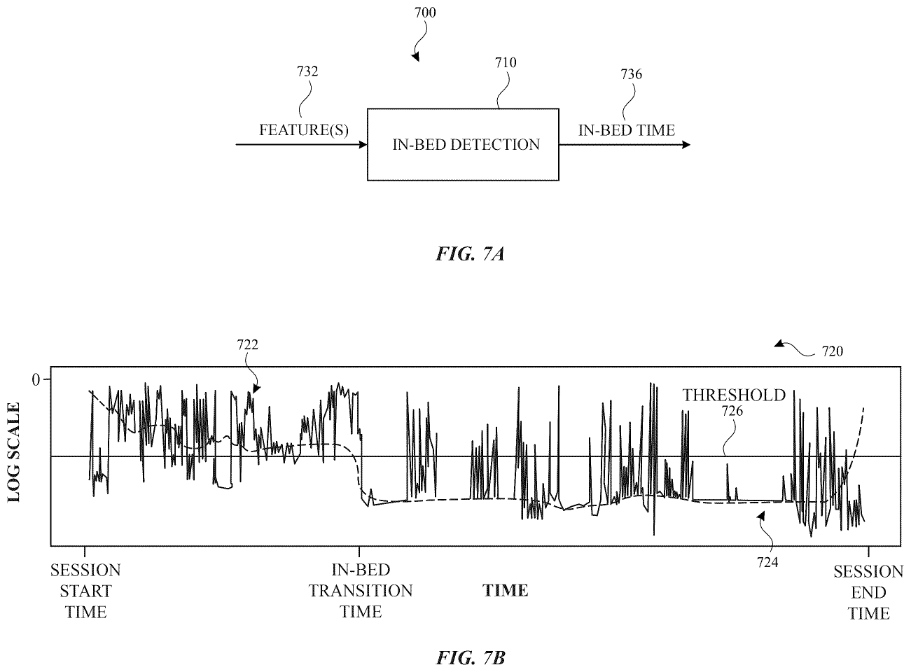

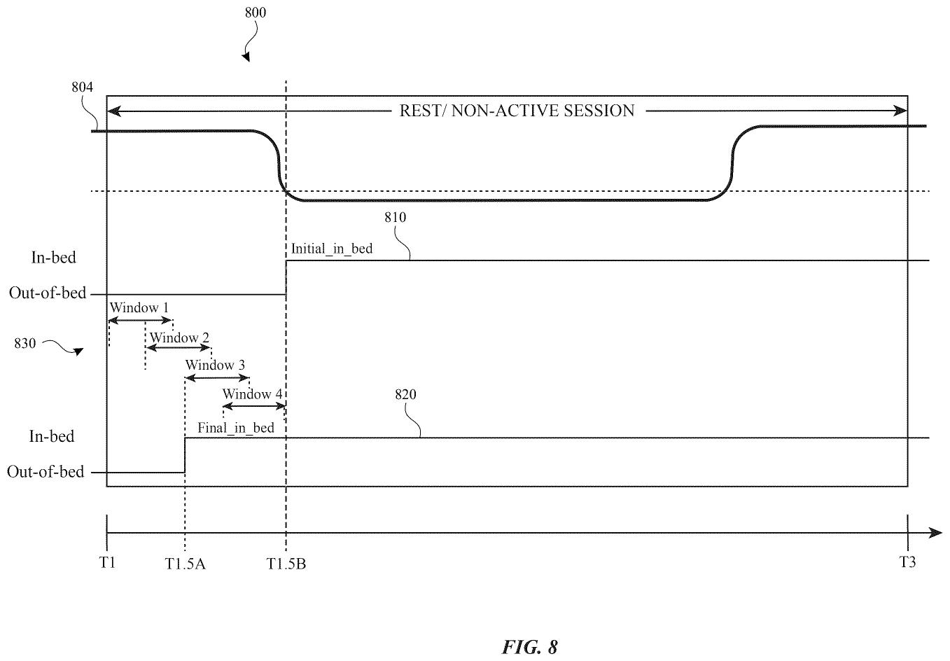

Generating the second mask can include one or more operations to determine a transition from an out-of-bed state to and in-bed state. A first operation can include a determine a transition from an out-of-bed state to and in-bed state using motion data. A second operation can include optionally determining an earlier transition from an out-of-bed state to and in-bed state using the output of ML model 408. FIGS. 7A-7B illustrate a block diagram 700 for in-bed transition detection and a plot 720 indicative of in-bed transition detection according to examples of the disclosure. FIG. 8 illustrates a timing diagram corresponding to the operations for generating the second mask according to examples of the disclosure.

In some examples, the generation of the second mask can include determining a transition time between the indication of a rest state (e.g., at T1 in FIG. 2B) and a detection that a user is “in bed” at some point during the sleep session (e.g., after T1 but before T2 in FIG. 2B). For example, one or more features extracted above for breathing disturbance classification can be used for in-bed detection by in-bed detection block 710. In-bed detection block 710 can estimate and output a time (e.g., an epoch corresponding to in-bed time 736) in the session in which the user transitions from being “out of bed” to being “in-bed.” The states of “out of bed” and “in-bed” may be defined as a function of movement (e.g., movement corresponding to sleep rather than rest or activity) rather than literally detecting whether the user is in a bed or not. In some examples, the one or more features 732 can include the maximum variance motion feature extracted by the feature extraction and first mask generator block 422. The maximum variance motion feature can be filtered and the transition to the “in-bed” state can be detected when the filtered feature drops below a threshold. In some examples, the threshold can be a user-specific threshold.

In some examples, a log 10 scale of the maximum variance motion feature can be used for in-bed detection (e.g., by taking the log base 10 of the maximum variance motion feature across the epochs of the session). For example, FIG. 7B illustrates a plot 720 with an example of a signal 722 corresponding to the log 10-scaled maximum variance motion feature between the session start time and the session end time (e.g., T0-T4). In some examples, this log 10-scaled maximum variance motion feature can be used to determine a user-specific threshold. The user-specific threshold can be set as the maximum between a default threshold (e.g., applicable to most users as defined by empirical data) and a threshold percentile (e.g., 55th percentile, 60th percentile, 65th percentile, etc.) of the log 10-scaled maximum variance motion feature. In some examples, the default threshold can be used without determining or using a user-specific threshold.

The log 10-scaled maximum variance motion feature can be filtered with a sliding window median-filter. The sliding window for in-bed detection can correspond to the duration of multiple epochs (e.g., 20, 50, 80, 100, 125, etc.). For the filtering, the session can be padded with zeros on both ends (indicative of high levels of activity in log base 10 scale). FIG. 7B illustrates signal 724 corresponding to the median-filtered log 10-scaled maximum variance motion feature (shown in dashed-line).

The epoch in which the median-filtered, log 10-scaled maximum variance motion feature falls below the threshold can be detected as the in-bed transition epoch. For example, FIG. 7B illustrates threshold 726, and the epoch at the in-bed transition time indicated where the median-filtered, log 10-scaled maximum variance motion feature crosses threshold 726. Similarly, timing diagram 800 FIG. 8 illustrates a simplified log 10-scaled maximum variance motion feature 804 during the rest/non-active session (e.g., between T1 and T3) that crosses the threshold indicating an initial in-bed time of T1.5B (between times T1 and T2). Additional details regarding FIG. 8 are described below with respect to a second operation to determine a transition from an out-of-bed state to and in-bed state.

In some examples, the output of the in-bed detection block 710 is an in-bed time 736 indicative of transition from a non-sleep to a sleep state. The in-bed or sleep state be no earlier than the start of the rest (non-active period) output by rest/active classifier 205, 405 or as indicated by the rest mask indicating a rest state and not an active state. In some examples, relying on motion data alone to determine the in-bed transition can be susceptible to delayed onset of the transition (e.g., due to user motion while falling asleep or in early sleep stages). To avoid missing potential breathing disturbances, another operation can be performed using the output of ML model 408 to detect an in-bed transition. For example, the when the pluralities of probabilities output by ML model 408 between the transition to the rest state from the non-rest state and the motion-based in-bed time 736 indicate a heightened-likelihood of a breathing disturbance, an earlier transition time can be determined. For example, one or more criteria can be used to evaluate a relatively longer (compared with the sliding window and the epochs for feature extraction) duration windows 830 (e.g., 1 minute, 2 minutes, 5 minutes, 10 minutes, etc.) of the likelihoods of a breathing disturbance. The windows 830 can be overlapping in time as well (e.g., 25%, 50%, etc. overlapping with adjacent windows). For example, FIG. 8 illustrates windows 830 labeled windows 1-4 (that are overlapping 50%) that are represented between T1 and T1.5B and/or including T1 and T1.5B, which can be evaluated for heighted-likelihood of a breathing disturbance (e.g., compared with the binary thresholding of the ML output for counting breathing disturbances). For example, the one or more criteria can include a criterion that is satisfied when a threshold percentile of the likelihoods of a breathing disturbance in a respective one of the relatively longer duration windows 830 is above a threshold likelihood. In some examples, the threshold percentile is 70^{th}, 80^{th}, 90^{th}, 95^{th}, etc. In some examples, the threshold likelihood is second likelihood threshold 458. In some examples, the threshold likelihood is 0.8, 0.825, 0.85, 0.875, 0.9, etc. When the one or more criteria are satisfied for this heighted-likelihood of the breathing disturbance for a respective relatively longer duration window between the initiation of the rest state and the in-bed time 736, an earlier transition time can be determined corresponding to this respective relatively longer duration window. For example, in the timing diagram 800, the plurality of likelihoods corresponding to the third window satisfy the one or more criteria for heightened-likelihood, and a final in-bed time of T1.5A (between times T1 and earlier than T1.5B) is determined. As shown in FIG. 8, the corresponding second mask 820 can includes “zeros” until the final in-bed time of T1.5A, and “ones” after the final in-bed time of T1.5A. In some examples, the earlier transition time can correspond to the start time of the respective relatively longer duration window. In some examples, each of the respective relatively longer duration windows 830 can be evaluated and the earliest of these windows can be used to determine the earlier in-bed transition time. In some examples, the evaluation begins for the respective relatively longer duration window corresponding to the transition to the resting state, and the evaluation continues forward in time until a respective relatively longer duration window with a heightened-likelihood is discovered or until no heightened-likelihood window is discovered after evaluating through to the respective relatively longer duration window including the in-bed transition (e.g., in-bed time 736). When no heightened-likelihood window is discovered, the initial in-bed time of T1.5B (between times T1 and T1.5B) is determined as the transition time. As shown in FIG. 8, the corresponding second mask 810 can includes “zeros” until the initial in-bed time of T1.5B, and “ones” after the initial in-bed time of T1.5B. The second mask can be a binary vector at the cadence of the sliding window representing signal quality states (e.g., with “ones” representing “an ‘in-bed’ or sleep state” state after the determined in-be transition (e.g., T1.5B unless an earlier transition time of T1.5A is indicated by heightened-likelihood of an earlier breathing disturbance) and “zeros” representing “an ‘out-of-bed’ or non-sleep resting” state before the determined in-bed transition).

Although rest/active classifier(s), the breathing disturbance classifier, and/or the signal quality classifier described herein use only motion data from a motion sensor (e.g., a 3-axis accelerometer), it is understood that, in some examples, one or more these classifiers include additional sensor inputs to improve some or all of these classifiers to improve the overall sleep/wake classification for the system. However, using only motion data can provide a low-power (and/or low-light) classification without the use of one or more additional sensors. In some examples, respiration features can be extracted from other sensors (e.g., using an optical sensor to extract respiration features (such as heart rate and heart rate variability features) from a photoplethysmography (PPG) signal or electrocardiogram (ECG) signal). In some examples, the breathing disturbance classifier can be augmented with data from one or more microphones (e.g., picking up audio indicative or sleep/wake or breathing characteristics, one or more pulse oximeters, one or more gyroscopes, etc. In some examples, a sensor strip (e.g., including one or more sensors such as piezoelectric sensors and/or proximity sensor(s)) on or in a bed can be used to detect respiration signals and/or motion signals for extraction of features (to improve performance and/or confidence of the rest/active classification, breathing disturbance classification, and/or quality check classification) and/or to detect in-bed conditions (e.g., for in-bed detection). In some examples, user inputs or states of the wearable device or another device (e.g., wearable device 100 and peripheral device 118) can be used as inputs as well. For example, user input to unlock/lock and/or to interact with the touchscreen or other input devices of the wearable device or a mobile phone or tablet computing device in communication with the wearable device can be used as indicators that a user is not in a sleep state (e.g., in a wake state and/or active state). This information can be used to correct incorrect classifications (e.g., false-positive breathing disturbance state classification) and/or can be used to forgo processing data to extract features and/or classify epoch when the contextual cues indicate an awake state.

As described herein, the processing of motion data for feature extraction can be done in real-time or in a cadence during operation. In some examples, the rest/active classifier can operate in real-time or in a cadence (e.g., during operation from T0 to T1 and/or from T2 to T3 illustrated in FIG. 2B). In some examples, the breathing disturbance classifier and/or the quality check classifier can be performed at the end of the session. In some examples, the feature extraction for breathing disturbance classifier and/or the quality check classifier can be performed in real-time or in a cadence during the session and the features can be assembled and/or processed by ML model at the end of the session (or in a cadence during the session).

As discussed above, aspects in of the present technology include the gathering and use of physiological information. The technology may be implemented along with technologies that involve gathering personal data that relates to the user's health and/or uniquely identifies or can be used to contact or locate a specific person. Such personal data can include demographic data, date of birth, location-based data, telephone numbers, email addresses, home addresses, and data or records relating to a user's health or level of fitness (e.g., vital signs measurements, medication information, exercise information, etc.).

The present disclosure recognizes that a user's personal data, including physiological information, such as data generated and used by the present technology, can be used to the benefit of users. For example, assessing a user's sleep conditions (e.g., to determine a user's rest/active state and/or sleep/wake state) may allow a user to track or otherwise gain insights about their health.

The present disclosure contemplates that the entities responsible for the collection, analysis, disclosure, transfer, storage, or other use of such personal data will comply with well-established privacy policies and/or privacy practices. In particular, such entities should implement and consistently use privacy policies and practices that are generally recognized as meeting or exceeding industry or governmental requirements for maintaining personal information data private and secure. Such policies should be easily accessible by users, and should be updated as the collection and/or use of data changes. Personal information from users should be collected for legitimate and reasonable uses of the entity and not shared or sold outside of those legitimate uses. Further, such collection/sharing should require receipt of the informed consent of the users. Additionally, such entities should consider taking any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices. The policies and practices may be adapted depending on the geographic region and/or the particular type and nature of personal data being collected and used.

Despite the foregoing, the present disclosure also contemplates examples in which users selectively block the collection of, use of, or access to, personal data, including physiological information. For example, a user may be able to disable hardware and/or software elements that collect physiological information. Further, the present disclosure contemplates that hardware and/or software elements can be provided to prevent or block access to personal data that has already been collected. Specifically, users can select to remove, disable, or restrict access to certain health-related applications collecting users' personal health or fitness data.

Therefore, according to the above, some examples of the disclosure are directed to a method. The method can comprise, at an electronic device including one or more motion sensors and processing circuitry: extracting, for each of a plurality of first epochs in a first session, a plurality of first features from first motion data from the one or more motion sensors; classifying pluralities of likelihoods of physiological events including classifying, for each of the plurality of first epochs in the first session, a plurality of likelihoods that the epoch includes one or more of the physiological events; applying one or more data masks to the pluralities of likelihoods for the plurality of first epochs; and determining a representation of a number of (e.g., occurrences of) the one or more of the physiological events in the first session. In some examples, determining a representation of the number one or more of the physiological events includes, in accordance with a determination that a first subset of the pluralities of likelihoods satisfies one or more first criteria, determining that the first subset corresponds to a first physiological event. In some examples, the one or more first criteria include a criterion that is satisfied when the first subset of the pluralities of likelihoods is greater than a first likelihood threshold and a criterion that is satisfied when the first subset corresponds to a consecutive period of time that is greater than a first time threshold. Additionally or alternatively to one or more of the examples disclosed above, in some examples, the method further comprises: downsampling motion data, including the first motion data, from the one or more motion sensors. Additionally or alternatively to one or more of the examples disclosed above, in some examples, the plurality of first features includes one or more frequency domain features. Additionally or alternatively to one or more of the examples disclosed above, in some examples, the plurality of first features includes a spectrogram.

Additionally or alternatively to one or more of the examples disclosed above, in some examples, classifying, for each of the plurality of first epochs in the first session, the plurality of likelihoods that the epoch includes the one or more physiological events comprises applying a machine learning model to the plurality of first features. Additionally or alternatively to one or more of the examples disclosed above, in some examples, the machine learning model includes a convolutional neural network in series with a recurrent neural network.

Additionally or alternatively to one or more of the examples disclosed above, in some examples, the plurality of likelihoods of a respective epoch of the plurality of first epochs includes one likelihood per interval.

Additionally or alternatively to one or more of the examples disclosed above, in some examples, the method further comprises extracting, for each of a plurality of second epochs in the first session, one or more second features from the first motion data from the one or more motion sensors. Additionally or alternatively to one or more of the examples disclosed above, in some examples, the one or more second features include one or more respiration features. Additionally or alternatively to one or more of the examples disclosed above, in some examples, the one or more second features include one or more movement features.

Additionally or alternatively to one or more of the examples disclosed above, in some examples, the method further comprises obtaining a first time corresponding to a transition from a non-rest state to a rest state, obtaining a second time corresponding to a transition from the rest state to the non-rest state, and generating a first mask of the one or more data masks including a representation of the rest state between the first time and the second time and a representation of the non-rest state before the first time and after the second time. Additionally or alternatively to one or more of the examples disclosed above, in some examples, the method can further comprise extracting, for each of a plurality of third epochs, one or more third features from second motion data from the one or more motion sensors and classifying the one or more third features to estimate a plurality of resting state confidences, each of the plurality of resting state confidences corresponding to one of the plurality of third epochs. In accordance with a determination that the plurality of resting state confidences satisfies one or more second criteria, the method can further comprise determining the first time corresponding to the transition from a non-rest state to a rest state. In accordance with a determination that the plurality of resting state confidences satisfies one or more third criteria, the method can further comprise determining the second time corresponding to the transition from the rest state to the non-rest state.

Additionally or alternatively to one or more of the examples disclosed above, in some examples, the method further comprises obtaining the first mask, obtaining at least a subset of the one or more second features including at least one of the one or more movement features, obtaining the pluralities of likelihoods for the plurality of first epochs, determining a transition from a pre-sleep state to a sleep state based on the first mask, at least the subset of the one or more second features including at least the one of the one or more movement features, and the pluralities of likelihoods for the plurality of first epochs, and generating a second mask of the one or more data masks including a representation of the pre-sleep state before the transition and a representation of the sleep state after the transition. Additionally or alternatively to one or more of the examples disclosed above, in some examples, the method further comprises determining, using the first mask and at least the subset of the one or more second features including at least the one of the one or more movement features, a transition from a first motion state to a second motion state. The second motion state corresponds to reduced motion relative to the first motion state. In accordance with a determination that one or more second criteria are satisfied, the method can further comprise determining the transition from the pre-sleep state to the sleep state as a start of the respective first epoch corresponding that satisfies the one or more second criteria. In accordance with a determination that the one or more second criteria are satisfied, the method can further comprise determining the transition from the pre-sleep state to the sleep state as the transition from the first motion state to the second motion state. The one or more second criteria can include a criterion that is satisfied when a likelihood characteristic of a respective first epoch of the plurality of first epochs before or corresponding to the transition from the first motion state to the second motion state is greater than a second likelihood threshold, greater than the first likelihood threshold.

Additionally or alternatively to one or more of the examples disclosed above, in some examples, the method further comprises obtaining at least a subset of the one or more second features including the one or more movement features and the one or more respiration features, and generating a third mask of the one or more data masks including a representation of a valid signal quality state and a representation of an invalid signal quality state.

Additionally or alternatively to one or more of the examples disclosed above, in some examples, the one or more motion sensors include one or more accelerometers. Additionally or alternatively to one or more of the examples disclosed above, in some examples, the one or more physiological event corresponds to one or more breathing disturbances. Additionally or alternatively to one or more of the examples disclosed above, in some examples, the one or more breathing disturbances correspond to stopping breathing for a threshold period of time.

Some examples of the disclosure are directed to a non-transitory computer readable storage medium. The non-transitory computer readable storage medium can store instructions, which when executed by an electronic device comprising processing circuitry, can cause the processing circuitry to perform any of the above methods. Some examples of the disclosure are directed to an electronic device comprising processing circuitry, memory, and one or more programs. The one or more programs can be stored in the memory and configured to be executed by the processing circuitry. The one or more programs can include instructions for performing any of the above methods.

Some examples of the disclosure are directed to an electronic device. The electronic device can comprise: one or more motion sensors (e.g., a multi-channel motion sensor) and processing circuitry coupled to the one or more motion sensors. The processing circuitry can be programmed to: extract, for each of a plurality of first epochs in a first session, a plurality of first features from first motion data from the one or more motion sensors; classify pluralities of likelihoods of physiological events including classifying, for each of the plurality of first epochs in the first session, a plurality of likelihoods that the epoch includes one or more of the physiological events; apply one or more data masks to the pluralities of likelihoods for the plurality of first epochs; and determine a representation of a number of the one or more of the physiological events in the first session. In some examples, determining a representation of the number one or more of the physiological events includes, in accordance with a determination that a first subset of the pluralities of likelihoods satisfies one or more first criteria, determining that the first subset corresponds to a first physiological event. In some examples, the one or more first criteria include a criterion that is satisfied when the first subset of the pluralities of likelihoods is greater than a first likelihood threshold and a criterion that is satisfied when the first subset corresponds to a consecutive period of time that is greater than a first time threshold.

Additionally or alternatively to one or more of the examples disclosed above, in some examples, the processing circuitry can be programmed to downsample motion data, including the first motion data, from the one or more motion sensors. Additionally or alternatively to one or more of the examples disclosed above, in some examples, the plurality of first features includes one or more frequency domain features. Additionally or alternatively to one or more of the examples disclosed above, in some examples, the plurality of first features includes a spectrogram.

Additionally or alternatively to one or more of the examples disclosed above, in some examples, classifying, for each of the plurality of first epochs in the first session, the plurality of likelihoods that the epoch includes the one or more physiological events comprises applying a machine learning model to the plurality of first features. Additionally or alternatively to one or more of the examples disclosed above, in some examples, the machine learning model includes a convolutional neural network in series with a recurrent neural network.

Additionally or alternatively to one or more of the examples disclosed above, in some examples, the plurality of likelihoods of a respective epoch of the plurality of first epochs includes one likelihood per interval.

Additionally or alternatively to one or more of the examples disclosed above, in some examples, the processing circuitry can be programmed to extract, for each of a plurality of second epochs in the first session, one or more second features from the first motion data from the one or more motion sensors. Additionally or alternatively to one or more of the examples disclosed above, in some examples, the one or more second features include one or more respiration features. Additionally or alternatively to one or more of the examples disclosed above, in some examples, the one or more second features include one or more movement features.

Additionally or alternatively to one or more of the examples disclosed above, in some examples, the processing circuitry can be programmed to obtain a first time corresponding to a transition from a non-rest state to a rest state, obtain a second time corresponding to a transition from the rest state to the non-rest state, and generate a first mask of the one or more data masks including a representation of the rest state between the first time and the second time and a representation of the non-rest state before the first time and after the second time. Additionally or alternatively to one or more of the examples disclosed above, in some examples, the processing circuitry can be programmed to extract, for each of a plurality of third epochs, one or more third features from second motion data from the one or more motion sensors and classify the one or more third features to estimate a plurality of resting state confidences, each of the plurality of resting state confidences corresponding to one of the plurality of third epochs. In accordance with a determination that the plurality of resting state confidences satisfies one or more second criteria, the processing circuitry can be programmed to determine the first time corresponding to the transition from a non-rest state to a rest state. In accordance with a determination that the plurality of resting state confidences satisfies one or more third criteria, the processing circuitry can be programmed to determine the second time corresponding to the transition from the rest state to the non-rest state.

Additionally or alternatively to one or more of the examples disclosed above, in some examples, the processing circuitry can be programmed to obtain the first mask, obtain at least a subset of the one or more second features including at least one of the one or more movement features, obtain the pluralities of likelihoods for the plurality of first epochs, determine a transition from a pre-sleep state to a sleep state based on the first mask, at least the subset of the one or more second features including at least the one of the one or more movement features, and the pluralities of likelihoods for the plurality of first epochs, and generate a second mask of the one or more data masks including a representation of the pre-sleep state before the transition and a representation of the sleep state after the transition. Additionally or alternatively to one or more of the examples disclosed above, in some examples, the processing circuitry can be programmed to determine, using the first mask and at least the subset of the one or more second features including at least the one of the one or more movement features, a transition from a first motion state to a second motion state. The second motion state corresponds to reduced motion relative to the first motion state. In accordance with a determination that one or more second criteria are satisfied, the processing circuitry can be programmed to determine the transition from the pre-sleep state to the sleep state as a start of the respective first epoch corresponding that satisfies the one or more second criteria. In accordance with a determination that the one or more second criteria are satisfied, the processing circuitry can be programmed to determine the transition from the pre-sleep state to the sleep state as the transition from the first motion state to the second motion state. The one or more second criteria can include a criterion that is satisfied when a likelihood characteristic of a respective first epoch of the plurality of first epochs before or corresponding to the transition from the first motion state to the second motion state is greater than a second likelihood threshold, greater than the first likelihood threshold.

Additionally or alternatively to one or more of the examples disclosed above, in some examples, the processing circuitry can be programmed to obtain at least a subset of the one or more second features including the one or more movement features and the one or more respiration features, and generate a third mask of the one or more data masks including a representation of a valid signal quality state and a representation of an invalid signal quality state.

Additionally or alternatively to one or more of the examples disclosed above, in some examples, the one or more motion sensors include one or more accelerometers. Additionally or alternatively to one or more of the examples disclosed above, in some examples, the one or more physiological event corresponds to one or more breathing disturbances. Additionally or alternatively to one or more of the examples disclosed above, in some examples, the one or more breathing disturbances correspond to stopping breathing for a threshold period of time.

Although examples of this disclosure have been fully described with reference to the accompanying drawings, it is to be noted that various changes and modifications will become apparent to those skilled in the art. Such changes and modifications are to be understood as being included within the scope of examples of this disclosure as defined by the appended claims.

1. An electronic device comprising:
one or more motion sensors; and
processing circuitry coupled to the one or more motion sensors, the processing circuitry programmed to:
extract, for each of a plurality of first epochs in a first session, a plurality of first features from first motion data from the one or more motion sensors;
classify pluralities of likelihoods of physiological events including classifying, for each of the plurality of first epochs in the first session, a plurality of likelihoods that the epoch includes one or more of the physiological events;
apply one or more data masks to the pluralities of likelihoods for the plurality of first epochs; and
determine a representation of a number of the one or more of the physiological events in the first session, including:
in accordance with a determination that a first subset of the pluralities of likelihoods satisfies one or more first criteria, including a criterion that is satisfied when the first subset of the pluralities of likelihoods is greater than a first likelihood threshold and a criterion that is satisfied when the first subset corresponds to a consecutive period of time that is greater than a first time threshold, determine that the first subset corresponds to a first physiological event.

2. The electronic device of claim 1, the processing circuitry further programmed to:
extract, for each of a plurality of second epochs in the first session, one or more second features from the first motion data from the one or more motion sensors.

3. The electronic device of claim 2, wherein:
the plurality of first features includes one or more frequency domain features and/or a spectrogram; and
the one or more second features include one or more respiration features and/or one or more movement features.

4. The electronic device of claim 1, the processing circuitry further programmed to:
obtain a first time corresponding to a transition from a non-rest state to a rest state;
obtain a second time corresponding to a transition from the rest state to the non-rest state; and
generate a first mask of the one or more data masks including a representation of the rest state between the first time and the second time and a representation of the non-rest state before the first time and after the second time.

5. The electronic device of claim 4, the processing circuitry further programmed to:
extract, for each of a plurality of third epochs, one or more third features from second motion data from the one or more motion sensors;
classify the one or more third features to estimate a plurality of resting state confidences, each of the plurality of resting state confidences corresponding to one of the plurality of third epochs;
in accordance with a determination that the plurality of resting state confidences satisfies one or more second criteria, determine the first time corresponding to the transition from a non-rest state to a rest state; and
in accordance with a determination that the plurality of resting state confidences satisfies one or more third criteria, determine the second time corresponding to the transition from the rest state to the non-rest state.

6. The electronic device of claim 5, the processing circuitry further programmed to:
obtain the first mask;
obtain at least a subset of one or more second features including at least one of one or more movement features;
obtain the pluralities of likelihoods for the plurality of first epochs;
determine a transition from a pre-sleep state to a sleep state based on the first mask, at least the subset of the one or more second features including at least the one of the one or more movement features, and the pluralities of likelihoods for the plurality of first epochs; and
generate a second mask of the one or more data masks including a representation of the pre-sleep state before the transition and a representation of the sleep state after the transition.

7. The electronic device of claim 6, the processing circuitry further programmed to:
determine, using the first mask and at least the subset of the one or more second features including at least the one of the one or more movement features, a transition from a first motion state to a second motion state, wherein the second motion state corresponds to reduced motion relative to the first motion state;
in accordance with a determination that one or more second criteria are satisfied, determine the transition from the pre-sleep state to the sleep state as a start of the respective first epoch corresponding that satisfies the one or more second criteria; and
in accordance with a determination that the one or more second criteria are satisfied, determine the transition from the pre-sleep state to the sleep state as the transition from the first motion state to the second motion state;
wherein the one or more second criteria including a criterion that is satisfied when a likelihood characteristic of a respective first epoch of the plurality of first epochs before or corresponding to the transition from the first motion state to the second motion state is greater than a second likelihood threshold, greater than the first likelihood threshold.

8. The electronic device of claim 7, the processing circuitry further programmed to:
obtain at least a subset of the one or more second features including the one or more movement features and one or more respiration features; and
generate a third mask of the one or more data masks including a representation of a valid signal quality state and a representation of an invalid signal quality state.

9. The electronic device of claim 1, wherein:
the one or more motion sensors include one or more accelerometers; and
the one or more physiological event corresponds to one or more breathing disturbances.

10. The electronic device of claim 9, wherein:
classifying, for each of the plurality of first epochs in the first session, the plurality of likelihoods that the epoch includes the one or more physiological events comprises applying a machine learning model to the plurality of first features; and
the machine learning model includes a convolutional neural network in series with a recurrent neural network.

11. A method comprising:
at an electronic device including one or more motion sensors and processing circuitry:
extracting, for each of a plurality of first epochs in a first session, a plurality of first features from first motion data from the one or more motion sensors;
classifying pluralities of likelihoods of physiological events including classifying, for each of the plurality of first epochs in the first session, a plurality of likelihoods that the epoch includes one or more of the physiological events;
applying one or more data masks to the pluralities of likelihoods for the plurality of first epochs; and
determining a representation of a number of the one or more of the physiological events in the first session, including:
in accordance with a determination that a first subset of the pluralities of likelihoods satisfies one or more first criteria, including a criterion that is satisfied when the first subset of the pluralities of likelihoods is greater than a first likelihood threshold and a criterion that is satisfied when the first subset corresponds to a consecutive period of time that is greater than a first time threshold, determining that the first subset corresponds to a first physiological event.

12. The method of claim 11, further comprising:
downsampling motion data, including the first motion data, from the one or more motion sensors.

13. The method of claim 11, further comprising:
extracting, for each of a plurality of second epochs in the first session, one or more second features from the first motion data from the one or more motion sensors, wherein:
the plurality of first features includes one or more frequency domain features and/or a spectrogram; and
the one or more second features include one or more respiration features and/or one or more movement features.

14. The method of claim 11, further comprising:
obtaining a first time corresponding to a transition from a non-rest state to a rest state;
obtaining a second time corresponding to a transition from the rest state to the non-rest state;
generating a first mask of the one or more data masks including a representation of the rest state between the first time and the second time and a representation of the non-rest state before the first time and after the second time;
extracting, for each of a plurality of third epochs, one or more third features from second motion data from the one or more motion sensors;
classifying the one or more third features to estimate a plurality of resting state confidences, each of the plurality of resting state confidences corresponding to one of the plurality of third epochs;
in accordance with a determination that the plurality of resting state confidences satisfies one or more second criteria, determining the first time corresponding to the transition from a non-rest state to a rest state; and
in accordance with a determination that the plurality of resting state confidences satisfies one or more third criteria, determining the second time corresponding to the transition from the rest state to the non-rest state.

15. The method of claim 14, further comprising:
obtaining the first mask;
obtaining at least a subset of one or more second features including at least one of one or more movement features;
obtaining the pluralities of likelihoods for the plurality of first epochs;
determining a transition from a pre-sleep state to a sleep state based on the first mask, at least the subset of the one or more second features including at least the one of the one or more movement features, and the pluralities of likelihoods for the plurality of first epochs;
generating a second mask of the one or more data masks including a representation of the pre-sleep state before the transition and a representation of the sleep state after the transition;
determining, using the first mask and at least the subset of the one or more second features including at least the one of the one or more movement features, a transition from a first motion state to a second motion state, wherein the second motion state corresponds to reduced motion relative to the first motion state;
in accordance with a determination that one or more second criteria are satisfied, determining the transition from the pre-sleep state to the sleep state as a start of the respective first epoch corresponding that satisfies the one or more second criteria; and
in accordance with a determination that the one or more second criteria are satisfied, determining the transition from the pre-sleep state to the sleep state as the transition from the first motion state to the second motion state;
wherein the one or more second criteria including a criterion that is satisfied when a likelihood characteristic of a respective first epoch of the plurality of first epochs before or corresponding to the transition from the first motion state to the second motion state is greater than a second likelihood threshold, greater than the first likelihood threshold.

16. The method of claim 15, further comprising:
obtaining at least a subset of the one or more second features including the one or more movement features and one or more respiration features; and
generating a third mask of the one or more data masks including a representation of a valid signal quality state and a representation of an invalid signal quality state.

17. The method of claim 11, wherein:
the one or more physiological event corresponds to one or more breathing disturbances; and
the one or more breathing disturbances correspond to stopping breathing for a threshold period of time.

18. A non-transitory computer readable storage medium storing instructions, which when executed by an electronic device including one or more motion sensors and processing circuitry, cause the processing circuitry to:
extract, for each of a plurality of first epochs in a first session, a plurality of first features from first motion data from the one or more motion sensors;
classify pluralities of likelihoods of physiological events including classifying, for each of the plurality of first epochs in the first session, a plurality of likelihoods that the epoch includes one or more of the physiological events;
apply one or more data masks to the pluralities of likelihoods for the plurality of first epochs; and
determine a representation of a number of the one or more of the physiological events in the first session, including:
in accordance with a determination that a first subset of the pluralities of likelihoods satisfies one or more first criteria, including a criterion that is satisfied when the first subset of the pluralities of likelihoods is greater than a first likelihood threshold and a criterion that is satisfied when the first subset corresponds to a consecutive period of time that is greater than a first time threshold, determining that the first subset corresponds to a first physiological event.

19. The non-transitory computer readable storage medium of claim 18, the instructions, when executed by the electronic device, further cause the processing circuitry to:
obtain a first time corresponding to a transition from a non-rest state to a rest state;
obtain a second time corresponding to a transition from the rest state to the non-rest state; and
generate a first mask of the one or more data masks including a representation of the rest state between the first time and the second time and a representation of the non-rest state before the first time and after the second time.

20. The non-transitory computer readable storage medium of claim 19, the instructions, when executed by the electronic device, further cause the processing circuitry to:
obtain the first mask;
obtain at least a subset of one or more second features including at least one of one or more movement features;
obtain the pluralities of likelihoods for the plurality of first epochs;
determine a transition from a pre-sleep state to a sleep state based on the first mask, at least the subset of the one or more second features including at least the one of the one or more movement features, and the pluralities of likelihoods for the plurality of first epochs;
generate a second mask of the one or more data masks including a representation of the pre-sleep state before the transition and a representation of the sleep state after the transition;
obtain at least a subset of the one or more second features including the one or more movement features and one or more respiration features; and
generate a third mask of the one or more data masks including a representation of a valid signal quality state and a representation of an invalid signal quality state.
